SEQUENCE LISTING (5 sequences)

SEQ ID NO: 1 - DNA, 20 bp, synthetic construct
  source 1..20: mol_type = other DNA
taatttctactgttgtagat

SEQ ID NO: 2 - DNA, 37 bp, synthetic construct
  source 1..37: mol_type = other DNA
ctgatgagtccgtgaggacgaaacgagtaagctcgtc

SEQ ID NO: 3 - DNA, 68 bp, synthetic construct
  source 1..68: mol_type = other DNA
ggccggcatggtcccagcctcctcgctggcgccggctgggcaacatgcttcggcatggcgaatgggac

SEQ ID NO: 4 - DNA, 57 bp, synthetic construct
  source 1..57: mol_type = other DNA
atttaaaaaaaaaaaaaaaaaaaaaaaaaaaaaaaaaaaaaaaaaaaaaaaggatcc

SEQ ID NO: 5 - DNA, 58 bp, synthetic construct
  source 1..58: mol_type = other DNA
atttaaaaaaaaaaaaaaaaaaaaaaaaaaaaaaaaaaaaaaaaaaaaaaaaggatcc

(19) United States
(12) Patent Application Publication
(10) Pub. No.: US 2023/0304026 A1
(43) Pub. Date: Sep. 28, 2023

(54) GUIDE EXPRESSED AS AN EXTENSION OF AN MRNA

(71) Applicant: Benson Hill, Inc., St. Louis, MO (US)

(72) Inventors: Zarir Erach Vaghchhipawala, St. Louis, MO (US); Lorena Beatriz Moeller, St. Louis, MO (US); Ross Alastair Johnson, St. Louis, MO (US); Scott Dour, St. Louis, MO (US); Farhad Moshiri, St. Louis, MO (US); Jaya Soneji, St. Louis, MO (US)

(73) Assignee: Benson Hill, Inc., St. Louis, MO (US)

(21) Appl. No.: 18/188,183

(22) Filed: Mar. 22, 2023

Related U.S. Application Data
(60) Provisional application No. 63/323,350, filed on Mar. 24, 2022.

(51) Int. Cl. C12N 15/82; C12N 9/22

(52) U.S. Cl. CPC C12N 15/8213; C12N 9/22; C12N 2310/20

(57) ABSTRACT

The present disclosure is directed to polynucleotides with a selection marker and a guide RNA module transcriptionally fused such that the selection marker and the genetic editing machinery are expressed concurrently. Accordingly, the selected cells are more likely to be genetically edited, such that the genetic editing efficiency is increased. The present disclosure also provides a method of transforming plant cells, a method of altering a target site in the genome of a plant cell, a method of increasing the fraction of genetically edited cells in a plant embryo following transformation using the polynucleotides.

### CROSS-REFERENCE TO RELATED APPLICATIONS

This application claims the benefit of U.S. Provisional Application No. 63/323,350, filed Mar. 24, 2022, which is incorporated by reference herein in its entirety.

### REFERENCE TO A SEQUENCE LISTING SUBMITTED AS AN XML FILE VIA PATENT CENTER

The application contains a sequence listing which is submitted herewith in electronically readable format. The Sequence Listing file was created on Mar. 22, 2023, is named B88552_0336_8_ Seq_List.xml and its size is 5.51 KB. The entire contents of the Sequence Listing are hereby incorporated by reference herein.

### FIELD OF THE INVENTION

The present disclosure relates to polynucleotides and methods for increasing the editing efficiency and homogeneity of genetically edited cells in a plant or plant part.

### BACKGROUND OF THE INVENTION

There is a delay between the time that a cell is transformed with polynucleotides encoding genome editing machinery and the time when the desired edit is made. This delay can reduce editing efficiency (e.g. the recovery of relatively large populations of edited cells). Currently the editing step does not seem to take place early after transformation, as evidenced by low frequencies of homogenously edited plants as a proportion of the incidence of transformed plants, whilst using nucleases for editing. There is likely a temporal disconnect between expression of the selectable marker vs. expression of nuclease and guide RNA in the transformed cell. If these processes were one coordinated event, the first cell after transformation would be edited too and would then give rise to a clonal edited cell population. Instead, editing seems to be an independent event over selection, and this scenario often leads to mosaic-edited plants, or, no editing at all since the editing machinery seems to be silent at the time of selection. A method for coordinated expression of the gene editing components along with selection markers could help to increase the editing efficiency and to create cell populations with a higher number of edited cells than traditional methods.

### SUMMARY

In one aspect, the present disclosure provides a polynucleotide comprising: (a) a gRNA transcriptionally fused to a marker gene, and (b) a gene encoding a nuclease. In some embodiments, the gRNA and the marker gene are operably linked to a first promoter, and the gene encoding the nuclease is operably linked to a second promoter.

In some embodiments, the gRNA is operably linked to the 3′ extension of marker gene. In some embodiments, the polynucleotide further comprises a spacer between the marker gene and the gRNA. In some specific embodiments, the spacer comprises a polyA, an HH ribozyme, an FnHP, or an Fn direct repeat, or a combination thereof.

In some embodiments, the marker gene is an antibiotic resistance gene. In one specific embodiment, the antibiotic resistance gene is a spectinomycin resistance gene.

In some embodiments, the marker gene and the gRNA are capable of concurrent expression. In some embodiments, the first and the second promoter are arranged end-to-end. In some embodiments, the nuclease is a Cpf1 nuclease.

In some embodiments, the marker gene, the gRNA and the nuclease express concurrently. In some embodiments, the polynucleotide comprises any one of the constructs of Table 3.

In some other aspects, the present disclosure provides a method of transforming a plant cell comprising the steps of: (i) introducing a polynucleotide into a plant cell; the polynucleotide is selected from the polynucleotide described herein, (ii) culturing the plant cell; and (iii) selecting for plant cells comprising the polynucleotide. In some embodiments, the selecting step comprises selecting for plant cells based on marker gene expression.

In some embodiments, the plant cell is cultured in media comprising at least one antibiotic. In some embodiments, the plant cell is from an early plant developmental stage. In some embodiments the nuclease cleaves a target site in the genome of the plant cell and a mutation is introduced into the cleaved target site.

In some embodiments, the fraction of cells with at least one mutation at said target site following introduction of the said polynucleotide is increased compared with plant cells following introduction of a control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

In some embodiments, the plant cell is from a plant selected from a group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

The present disclosure further provides a method of altering a target site in the genome of a plant cell, comprising the steps of: (i) introducing a polynucleotide into the plant cell; the polynucleotide is selected from the polynucleotide as described herein; (ii) culturing the plant cell for a sufficient duration; (iii) selecting for plant cells comprising the polynucleotide; such that the target site is altered.

In some embodiments, the plant cell is cultured in media comprising at least one antibiotic. In some embodiments, the selecting step comprises selecting for plant cells based on marker gene expression. In some embodiments, the plant cell is from an early plant development stage.

In some embodiments, the marker gene, the gRNA and the nuclease express concurrently. In some embodiments, the fraction of cells with at least one altered target site following introduction of the said polynucleotide is increased compared with plant cells following introduction of a proper control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

In some embodiments, the plant cell is from a plant selected from a group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In a further aspect, the present disclosure provides a method of increasing the fraction of genetically edited cells in a plant embryo following transformation, said method comprising introducing into the plant embryo a polynucleotide comprising: (a) a gRNA transcriptionally fused to a marker gene, and (b) a gene encoding a nuclease. In some embodiments, the gRNA and the marker gene are operably linked to a first promoter, and the gene encoding the nuclease is operably linked to a second promoter. The first promoter and second promoter can be selected for coordinated expression (e.g., concurrent) of the corresponding operably linked nucleic acid sequences. In particular embodiments, the genes operably linked to the first promoter and the genes operably linked to the second promoter express concurrently, such as at the same time or during the same time period. Concurrent expression need not start and end at exactly the same time, but encompasses overlapping expression. In some embodiments, the fraction of genetically edited cells following introduction of said polynucleotide is greater than the fraction of genetically edited cells following introduction of a proper control polynucleotide.

In certain embodiments, the polynucleotide is any polynucleotide described herein. In certain embodiments, the marker gene, the gRNA, and the nuclease express concurrently. In certain embodiments, the fraction of genetically edited cells following introduction of said polynucleotide is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

In various embodiments, the plant cell is from a plant selected from a group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

The present disclosure further provides a plant comprising a polynucleotide comprising: (a) a gRNA transcriptionally fused to a marker gene, and (b) a gene encoding a nuclease. In various embodiments, the gRNA and the marker gene are operably linked to a first promoter, and the gene encoding the nuclease is operably linked to a second promoter.

In some embodiments, the genome of the plant comprises an alteration at a target site. In certain embodiments the polynucleotide is any polynucleotide described herein.

In some embodiments, the marker gene, the gRNA and the nuclease express concurrently. In some embodiments, the fraction of cells with at least one alteration at a target site is increased compared with a plant comprising a proper control polynucleotide. In some embodiments, the fraction of cells with at least one alteration at a target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

In some embodiments, the plant is selected from a group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

### BRIEF DESCRIPTION OF DRAWINGS

FIGS. 1A-1B show guide RNAs expressed as a 3′ extension downstream of a GFP CDS display normal GFP expression and editing at target sites when expressed in yellow pea protoplast cells.

FIG. 1A shows the expression of GFP of different constructs in yellow pea protoplast cells;

FIG. 1B shows the editing efficiency of target sites by different constructs in yellow pea protoplast cells.

FIG. 2 presents the conceptual design of the constructs. The upper panel shows the CP4-led strategy for driving selection of first transformed cell. The lower panel expands on this strategy to deliver auxiliary factors that promote desirable processes, such as the division of the first edited cells (as mediated by morphogens), HDR editing outcomes, or non-invasive base change strategies like Base-Editors.

FIGS. 3A-3E depict different designs of single transcription unit (STU). FIG. 3A is 135856 (2× FnDR(35)-FAD2B-2); FIG. 3B is 135856-BamHI in place of SwaI (135885); FIG. 3C is 135856 with polyA (50) (135886); FIG. 3D is 135856-withRz-flanked FADB2-2 (135887);

FIG. 3E is 135856 with polyA (50)+Rz-flanked FAD2B-2 (135888).

FIG. 4 shows the editing efficiency of control (no single transcription unit), and different STU constructs including STU-DR-Guide V1, STU-DR-Guide V2, STU+polyA50-DR-Guide, STU+RZ-DR-Guide, and STU+polyA50+RZ-DR-Guide.

FIG. 5 shows the design of STU vector STU+PolyA50+Rz-DR-Guide for soy transformation.

FIGS. 6A-6D show the NGS data indicating the repaired edits and co-editing of both copies with constructs based on SPCR-STU concept. Targeting the PDS1 and PDS2 genes in Soybean. FIG. 6A shows the results of NGS screening of highly edited plants samples from a prior ddPCR screening shows % total edited reads for PDS1gene (blue bar), % Top edit reads (orange bar) and % second top edit reads (gray bar) from plant samples. Each bar is a fraction of total qualified reads; FIG. 6B shows the results of NGS screening of highly edited plants samples from a prior ddPCR screening shows % total edited reads for PDS2 gene (blue bar), % Top edit reads (orange bar) and % second top edit reads (gray bar) from plant samples. Each bar is a fraction of total qualified reads; FIG. 6C shows the total edited reads from both genes PDS1 (blue) and PDS2 (orange) from each highly edited soybean event, illustrating the multiplex editing power of the technology; FIG. 6D shows the % Top edit at both genes PDS1 (blue bar) and PDS2 (orange bar) from each highly edited soybean event, illustrating editing homogeneity in those events at both targets.

FIGS. 7A-7F present the schematic designs of STU constructs. FIG. 7A shows the schematic design of control construct 136362; FIG. 7B shows the schematic design of control construct 137458; FIG. 7C shows the schematic design of constructs 137088, 137089 and 136008; FIG. 7D shows the schematic design of constructs 137090, 137091 and 137092; FIG. 7E shows the schematic design of constructs 137093, 137094 & 137095.

FIG. 8 reports the ddPCR data indicating the target editing efficiency of different constructs in soybean cells.

FIG. 9 presents the shooting frequency of N35D950S using STU constructs, including constructs 135909, 137088, 137090, 137093, 136362, 137458, 137095, 137092, 136008, 137094, 137091 and 137089.

### DETAILED DESCRIPTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

A “plant” refers to a whole plant, any part thereof, or a cell or tissue culture derived from a plant, comprising any of: whole plants, plant components or organs (e.g., leaves, stems, roots, embryos, pollen, ovules, seeds, grains, leaves, flowers, branches, fruit, pulp, juice, kernels, ears, cobs, husks, stalks, root tips, anthers, etc.), plant tissues, plant cell tissue cultures from which plants can be regenerated, plant calli, plant clumps, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants comprising the introduced polynucleotides are also within the scope of the invention. Further provided is a processed plant product (e.g., extract) or byproduct that retains one or more polynucleotides disclosed herein.

Commonly known plant species includes, without limitations, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

Plant cells possess nuclear, plastid, and mitochondrial genomes. The polynucleotides and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

“Mosaicism” or “genetic mosaicism”, as used herein, refers to a condition in multicellular organisms in which a single organism possesses more than one genetic line as the result of genetic mutation, e.g., introduced by the polynucleotide described herein. For example, the degree of mosaicism of a plant refers to the population of cells within the reference plant that contain a specific mutation or a mutation at a specific site. A plant with low mosaicism has more cells with the mutation, and a plant with high mosaicism has a higher degree of heterogeneity of cells with the mutation. Thus, by decreasing the degree of mosaicism of a given mutation or edit within a plant or plant part, the plant or plant part has less of a mixed population of edited/mutated cells (i.e., less heterogeneity) compared to a control cell. The degree of mosaicism can be quantitative accessed via a variety of methods known in the art, for example, based on the assessment of Copy-Number Variant (CNV) deletions (Liu et al., Curr Protoc Hum Genet., 2020; 106(1):e99. doi: 10.1002/cphg.99)

As used herein, the term “early development stage” of a plant or “early plant” developmental stage refers to the germination stage and the seedling stage of a plant's development. A seed starts to germinate in favorable conditions in response to environmental stimuli such as light, temperature, soil components (especially nitrate), and the molecular mechanisms of a response that have been well characterized. Germination is a complex process during which the mature seed resumes growth and shifts from a maturation- to germination-driven program of development and subsequent seedling growth. By definition, the germination of a seed starts with the uptake of water and is completed when the radicle protrudes from the covering structures. In the case of the germination of a monocotyledonous plant seed, the coleorhiza is the first part to grow out of the seed coat, whereas during the germination of a dicotyledonous plant seed, the radicle grows out of the seed coat first. In both groups, the progress of germination is strictly related with the water uptake rate. Initially, there is a rapid imbibition of water by a dry seed (phase I) until the seed tissues are fully hydrated. This is followed by a limited water uptake during phase II, whereas in phase III, there is an increase of water uptake that is related to the completion of germination. The most important is phase II, which is associated with various cellular and biochemical events such as DNA repair and the translation of stored as well as newly synthesized mRNAs. Phase II is characterized by both increased metabolic and cellular activity. At the germination stage, the decision of embryo cells to re-enter the cell cycle or to remain arrested is crucial in determining seedling formation. The cell cycle, which is arrested in a quiescent seed, is reversed during germination (Wolny et al., Int J Mol Sci., 2018; 19(10): 2916).

The seedling stage is one of the most critical phases during a plant's life history. Seedling survival not only exerts an important influence on the size, persistence, and genetic variability of plant populations. One of ordinary skills in the art would understand seedling is broadly defined, for example, as a young plant still reliant on food reserves stored in the seed (Hanley et al., New Phytologist, 2004, 163: 61-66).

The “editing efficiency” of a genetic editing agent or system, as used herein, reflects the strength of the editing agent or system for altering the target site. The editing efficiency can be quantitatively accessed via, for example, measuring the ratio of cells edited, or the counts of edited target site.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term “genome” includes both the genes (the coding regions), the non-coding DNA and, if present, the genetic material of the mitochondria and/or chloroplasts, or the genomic material encoding a virus, or part of a virus. The “genome” or “genetic material” of an organism usually consists of DNA, wherein the genome of a virus may consist of RNA (single-stranded or double-stranded).

As used herein, “transcriptionally fused” refers to more than one coding sequence that are transcribed onto the same mRNA. Accordingly, in certain embodiments, the transcriptionally fused coding sequences or genes can be controlled by a single promoter.

A “morphogen”, as used herein, refers to a molecule that is involved in organogenesis or embryogenesis. Having “morphogen activity” or “morphogenic activity”, as used herein, refers to having a function or an activity in the process of organogenesis, embryogenesis, or early development of a plant or plant part. Morphogens can be employed with, or in lieu of, exogenous phytohormones to enhance regeneration, whilst selecting for transformants using resistance markers. Morphogens more directly stimulate transformed cells to regenerate into plants. In maize transformation, advanced morphogen expression approaches have enabled more stable regenerated transformants plants being produced across and within transformed explants, with fewer inputs of skilled labor time and explant inputs, sometimes enabling transformation of recalcitrant lines. Exemplary morphogens include ISOPENTYL TRANSFERASE (IPI) and WUSCHEL 2 (WUS2), e.g., maize-derived WUS2 (ZmWUS2). In some embodiments, the marker gene encodes a morphogen.

The term “terminator”, as used herein, refers to DNA sequences located downstream, i.e. in 3′ direction, of a coding sequence and can include a polyadenylation signal and other sequences, i.e. further sequences encoding regulatory signals that are capable of affecting mRNA processing and/or gene expression. The polyadenylation signal is usually characterized in that it adds polyA nucleotides at the 3′-end of an mRNA precursor.

The term “indel” or “INDEL” as used herein means and insertion and/or deletion in the genome of an organism, or in the genomic material of a cell or cellular system of interest.

The terms “guide RNA”, “gRNA”, “single guide RNA”, or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. A tracr and a crRNA moiety, if present as required by the respective CRISPR polypeptide, thus do not necessarily have to be present on one covalently attached RNA molecule, yet they can also be comprised by two individual RNA molecules, which can associate or can be associated by non-covalent or covalent interaction to provide a gRNA according to the present disclosure. In the case of single RNA-guided endonucleases like Cpf1, for example, a crRNA as single guide nucleic acid sequence might be sufficient for mediating DNA targeting.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples include insertions, deletions, duplications, substitutions, inversions, and translocations of any nucleic acid sequence, regardless of how the mutation is brought about and regardless of how or whether the mutation alters the functions or interactions of the nucleic acid. For example, and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, guide RNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with a mutation” or “plant part with a mutation” or “plant cell with a mutation” or “plant genome with a mutation” refers to a plant or plant part or plant cell or plant genome that contains a mutation (e.g., an insertion, a substitution, or a deletion) described in the present disclosure.

“Genome editing” or “gene editing” as used herein refers to a type of genetic engineering by which one or more mutations (e.g., insertions, substitutions, deletions, modifications) are introduced at a specific location of the genome. “Editing reagents”, as used herein, refers to a set of molecules or a construct comprising or encoding the molecules for introducing one or more mutations in the genome. Exemplary editing reagents include a nuclease and a guide RNA. For example, a CRISPR (clustered regularly interspaced short palindromic repeats) system includes a CRISPR nuclease [e.g., CRISPR-associated (Cas) endonuclease or a variant thereof, such as Cas12a] and a guide RNA. A CRISPR nuclease associates with a guide RNA that directs nucleic acid cleavage by the associated endonuclease by hybridizing to a recognition site in a polynucleotide. The guide RNA contains a direct repeat and a guide sequence, which is complementary to the target recognition site. In certain embodiments, the CRISPR system further contains a tracrRNA (trans-activating CRISPR RNA) that is complementary (fully or partially) to the direct repeat sequence present on the guide RNA.

“Genome editing” or “gene editing” as used herein refers to a type of genetic engineering by which one or more mutations (e.g., insertions, substitutions, deletions, modifications) are introduced at a specific location (i.e. target site) of the genome. “Editing reagents”, as used herein, refers to a set of molecules or a construct including or encoding the molecules for introducing one or more mutations in the genome. Exemplary editing reagents include a nuclease and a guide RNA. For example, a CRISPR (clustered regularly interspaced short palindromic repeats) system includes a CRISPR nuclease [e.g., CRISPR-associated (Cas) endonuclease or a variant thereof, such as Cas12a] and a guide RNA. A CRISPR nuclease associates with a guide RNA that directs nucleic acid cleavage by the associated endonuclease by hybridizing to a recognition site in a polynucleotide. The guide RNA includes a direct repeat and a guide sequence, which is complementary to the target recognition site. In certain embodiments, the CRISPR system further includes a tracrRNA (trans-activating CRISPR RNA) that is complementary (fully or partially) to the direct repeat sequence present on the guide RNA. A “TALEN” nuclease is an endonuclease including a DNA-binding domain containing a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, the terms “nuclease” and “endonuclease” are used interchangeably to refer to naturally-occurring or engineered enzymes, which cleave a phosphodiester bond within a polynucleotide chain. The cleavage could be a single strand cleavage or a double strand cleavage. In certain embodiments, the nuclease lacks cleavage activity and is referred to as nuclease dead. In various embodiments, the nuclease has nickase activity.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct contains an artificial combination of nucleic acid fragments, including, without limitation, regulatory molecules and polynucleotides that are not found together in nature. For example, a recombinant DNA construct may contain regulatory molecules and polynucleotides that are derived from different sources, or regulatory molecules and polynucleotides derived from the same source and arranged in a manner different than that found in nature. Such a construct may be used by itself or may be used in conjunction with a vector. In specific embodiments, a recombinant DNA construct or expression cassette includes a promoter operably linked to a polynucleotide of interest, wherein the promoter is heterologous to the polynucleotide of interest.

An expression construct can permit transcription of a particular nucleotide sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” is intended to mean a functional linkage between two or more elements. For example, an operable linkage between a promoter of the present invention and a nucleic acid molecule is a functional link that allows for expression of the nucleic acid molecule. Operably linked elements may be contiguous or non-contiguous. When used to refer to the joining of two protein coding regions, by operably linked is intended that the coding regions are in the same reading frame. The cassette may additionally contain at least one additional polynucleotide for co-transforming into the plant. Alternatively, the additional polynucleotide(s) can be provided on multiple expression cassettes or DNA constructs. Such an expression cassette or construct is provided with a plurality of restriction sites and/or recombination sites for insertion of the heterologous nucleotide sequence of interest to be under the transcriptional regulation of the promoter regions of the invention. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a polynucleotide, a peptide, a protein, or a molecule refers to activity of a gene, a polynucleotide, a peptide, a protein, or a molecule. For example, the function of a morphogen may be assessed by developmental phenotypes of plants or plant parts comprising the morphogen, e.g., number and form of shoot formation in the plants or plant parts.

As used herein, the term “expression” or “expressing” refers to the transcription and/or translation of a particular nucleic acid sequence driven by a promoter.

“Introduced” or “introducing” in the context of inserting a nucleic acid molecule (e.g., a DNA construct comprising a promoter molecule and a polynucleotide sequence of interest) into a cell, a plant, or a plant part means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a proper control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” or “enhanced” or “enhancing” or “enhance” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%) positive change in the parameter from a proper control, e.g., an established normal or reference level of the parameter, or an established standard control.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may contain a nucleic acid sequence which is identical or partially homologous to an endogenous nucleic acid sequence of the plant.

Computer implementations of these mathematical algorithms for comparison of sequences to determine sequence identity include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection. Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

When genome editing machinery is introduced into eukaryotic cells, there is often a delay between the time that a cell is transformed with polynucleotides encoding genome editing machinery and the time when the desired edit is made. This delay can reduce editing efficiency and decrease the homogeneity of the transformed plant.

The instant disclosure provides polynucleotides comprising a gRNA module transcriptionally fused to a marker gene along with a gene encoding a nuclease. When introduced into a eukaryotic cell, the polynucleotides are capable of reducing the delay between transformation and editing, and thereby increasing the efficiency of recovering cells containing a desired edit. In certain embodiments, these elements coordinate the step of editing with the process of selection such that they occur substantially at the same time, or at least reduce the delay that typically manifests between transformation and editing. Benefits of this system include an increase in editing efficiency, a decrease in the degree of mosaicism of the transformed plant, reduced experiment inputs for obtaining complex editing outcomes as compared to a control where the nuclease, the guide, and the selection marker are expressed separately, and a decrease in vector complexity and size. In some embodiments, the first transformed cell becomes the first edited cell and gives rise to a clonal population of homogenously edited cells resulting in a mono or bi-allelic edit throughout an organism, such as a plant, and not a mosaic edit pattern where different tissues have different edits arising at different time points.

In certain embodiments, the gRNA module can be expressed concurrently with a marker gene such as spectinomycin or CP4. In certain embodiments, the gRNA module, the marker gene, and the nuclease can be expressed concurrently. Thus, by cultivating the cells under appropriate selection pressures, the only surviving cells are those that express the marker gene. Because the gRNA module is operably linked to the same promoter, e.g., a constitutive promoter, as the gene encoding the marker it is also expressed when the marker gene is expressed. Therefore, only those cells that also express gRNA can survive under selective pressure, thereby increasing transformation efficiency. In certain embodiments, these steps occur in the same cell, i.e., the first transformed and edited cell.

The marker gene of the instant disclosure expressed concurrently with the gRNA is not limited to selectable markers, i.e., antibiotics. Instead, it can be any gene of interest to one of ordinary skill of art. For example, the polynucleotide concurrently expressed with the gRNA could be a sequence of DNA encoding a protein that alters the development of a plant, e.g. morphogens, enhancers of HDR, RNAi derivatives, etc. Substantially any gene one or ordinary skill in the art desires to be expressed early in the plant life cycle would also be of particular utility.

In other aspects, the instant disclosure provides methods using the polynucleotide for inserting a mutation in the genome of a cell. In one aspect, the instant disclosure provides methods of transforming a plant cell including the steps of (i) introducing the polynucleotide discussed herein into a plant cell, (ii) culturing the plant cell, and (iii) selecting for plant cells comprising the polynucleotide. In another aspect, the instant disclosure further provides methods of altering a target site in the genome of a plant cell including the steps of (i) introducing the polynucleotide into the plant cell, (ii) culturing the plant cell for a sufficient duration, and (iii) selecting for plant cells comprising the polynucleotide such that the target site is altered. In another aspect, the instant disclosure provides methods of increasing the fraction of genetically edited cells in a plant embryo following transformation, comprising introducing into the plant embryo the polynucleotide comprising, such that the fraction of genetically edited cells following introduction of the polynucleotide is greater than the fraction of genetically edited cells following introduction of a proper control polynucleotide. In yet another aspect, the instant disclosure provides plants having the polynucleotide.

The instant disclosure demonstrates that coordinating the expression of the selection marker, the target guide, and the nuclease together in a temporal manner, editing can be increased in those early transformed cells or enriching the population of regenerated transgenic cells to select for those that are edited. Accordingly, the selected cells are likely to be edited, and give rise to a homogeneous or substantially homogeneous lineage of cells.

Methods and compositions described herein can be useful also to deliver factors needed for editing, such as proteins involved in the five major DNA repair pathways (base excision repair (BER), nucleotide excision repair (NER) Micro-homology mediated End Joining (MMEJ), Homologous Recombination (HR) and Non-Homologous End Joining (NHEJ)) or base editing in a compact vector format to enhance the desired editing outcomes. For example, the RAD54 gene could be employed for enhancing the incidence of HR for transferred DNA molecules (Shaked et al., PNAS, 2015, 102 (34) 12265-12269), whilst providing a scaffold for guide RNA expression from a 3′ extended position downstream. Further provided is the use of morphogen-led gRNA expression for future non-selection/non-transgenic genome editing event generation. As a means to ensure high editing frequencies, we would seek to combine the latter morphogen-based approach with high-expression, such as for the ISOPENTENYLTRANSFERASE or SHOOTMERISTEMLESS morphogenic genes (Maher et al., Nat Biotechnol. 2020, 38(1):84-89), rather than those only desired for limited expression like WUSCHEL (Hoerster et al., In Vitro Cellular & Developmental Biology—Plant, 2020, 56, 265-279).

Another avenue of approach of using the instant disclosure could be a simulfrost blanket plantstaneous RNA-interference (RNAi) and editing approach, where downregulation of certain genes is beneficial for improving editing frequencies (Ye et al., Cell Discovery, 2018, 4:46). This approach would employ the replacement of a protein-coding sequence with an RNAi cassette upstream of the DR-guide sequences or using small target sequences to generate siRNA. Another application would be to deliver a repair template encoded through RNA for HDR (Butt et al., Front Plant Sci., 2017, 8:1441).

### III. Polynucleotides Containing 3′ Extended gRNA

In specific embodiments, the present disclosure provides a polynucleotide containing (a) a gRNA transcriptionally fused to a marker gene; (b) a gene encoding a nuclease such that the gRNA and the marker gene are operably linked to a first promoter, and the gene encoding the nuclease is operably linked to a second promoter. Exemplary constructs are provided in Table 3. In some embodiments, the polynucleotide comprises, operably linked from 5′ to 3′, a constitutive promoter, marker gene, polyA site, HH Ribozyme site, Fn direct repeat site, nucleic acid sequence encoding a gRNA, HDV ribozyme site (“HDV”), and a terminator, along with from 5′ to 3′, a constitutive promoter, a gene encoding a nuclease, and a terminator, wherein the constitutive promoters are positioned end-to-end. In one specific embodiment, the construct includes from 5′ to 3′ the AtUBI10p promoter, SpcR marker gene, PolyA site, HH ribozyme and Fn direct repeat sites (collectively “Rz” below), gRNA encoding sequence, HDV sequence, and NOSt terminator sequence. In another specific embodiment, the construct includes from 5′ to 3′ AtUBI10p, SpcR, PolyA, Rz, a gRNA-encoding sequence, HDV sequence, and NOSt. In another specific embodiment, the construct includes from 5′ to 3′ 2×35S Enhancer, AtUBI10p, SpcR, PolyA, Rz, a gRNA-encoding sequence, HDV sequence, and NOSt. In another specific embodiment, the construct includes from 5′ to 3′ GmScream M4 promoter, SpcR gene, PolyA, Rz, a gRNA-encoding sequence, and NOSt. In various specific embodiments, the constructs above further include from 5′ to 3′ PsUBI3 promoter, nuclease encoding sequence, NLS, and HSP terminator, wherein the promoters are positioned end-to-end.

The gRNA and the marker gene can be transcriptionally fused. As used herein, “transcriptionally fused” refers to more than one coding sequence that are transcribed onto the same mRNA. Accordingly, in various embodiments, the gRNA and the marker gene are transcribed onto the same mRNA. The gRNA and the marker gene, in various embodiments, can be controlled by the same first promoter. In many embodiments, the gRNA and the marker gene express concurrently in a same cell, i.e., a plant cell. The term “express concurrently”, “expressed concurrently”, and “concurrent expression”, as used herein, refers to two or more genes expressing at about the same time, such that the products of the genes co-exist in the same cell.

In some embodiments, the polynucleotide includes a single gRNA. In other embodiments, the polynucleotide includes multiple gRNAs each of which targeting a different target site. In some other embodiments, the instant disclosure provides a mixture of multiple polynucleotides. In some embodiments, any one of the multiple polynucleotides includes one or more than one gRNAs, each of which targets a different target site.

The nuclease encoded by the polynucleotide can be any nuclease described herein. In some embodiments, the cleavage could be a single strand cleavage or a double strand cleavage. A nuclease can be a nickase, an endonuclease, a meganuclease, or a nuclease fusion. For example, a Cas12a (Cpf1) endonuclease coupled with a guide RNA (guide RNA) designed against the genomic sequence of interest can be used (i.e., a CRISPR-Cas12a system). Alternatively, a Cas9 endonuclease coupled with a guide RNA designed against the genomic sequence of interest (a CRISPR-Cas9 system), or a Cms1 endonuclease coupled with a guide RNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al., Nat Rev Microbiol, 2020, 18:67-83)) with their corresponding guide RNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of a polynucleotide, thereby modulating the function of the polynucleotide (Piatek et al., Plant Biotechnol, 2015, J 13:578-589). In some embodiments, the nuclease encoded by the coding sequence of the DNA construct is a CRISPR-associated Cas endonuclease. In specific embodiments, the CRISPR nuclease is a Cas12a nuclease, herein used interchangeably with a Cpf1 nuclease. In a specific embodiment, the Cas12a nuclease is a McCpf1 nuclease, e.g., a Mc.2Cpf1 2C-NLS nuclease. In some embodiments, the nuclease is further operably linked to one or more nuclear localization sequences (NLSs) and/or one or more epitope tags. In certain embodiments, the nuclease lacks cleavage activity and is referred to as dead nuclease. In various embodiments, the nuclease has nickase activity. One of ordinary skill in the art can choose any nuclease according the gRNA in order to maximize the editing efficiency. In some preferred embodiments, the nuclease is Cpf1 nuclease.

In some embodiments, the gRNA is arranged to the 3′ end of the marker gene. The marker gene can be directly flanked by the gRNA at the 3′ end, or separated by a spacer sequence. In the embodiments where the gRNA and the 3′ of the marker gene are separated by a spacer, the spacer sequence can be about 15 bp, about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 300 bp, about 400 bp, about 500 bp, about 600 bp, about 700 bp, about 800 bp, about 900 bp, or about 1000 bp in length. In some embodiments, the spacer is any one of polyA, hammerhead ribozyme sequence, Fn direct repeat (FnDR), hepatitis delta virus ribozyme sequence, FnHP, or a combination thereof. In some embodiments, the FnDR is about 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 35 nt, 37 nt, 38 nt, 39 nt, or about 40 nt in length. In one particular embodiment, the FnDR is about 25 nt in length. In another specific embodiment, the FnDR is about 35 nt in length. In some embodiments, the FnHP contains the sequence of AAATTA.

As used herein an “Fn direct repeat” or “Fn mature” sequence refers to a sequence that is recognized and cleaved by the Cpf1 (Cas12a) nuclease. In certain embodiments, the Fn direct repeat is a 19 base pair direct repeat identified from Francisella novicida containing the sequence of TAATTTCTACTGTTGTAGAT (SEQ ID NO: 1) (Swiat et al., Nucleic Acids Res., 2017; 45(21):12585-12598).

A “hammerhead ribozyme sequence” or “HH ribozyme” or “HH sequence”, as used herein, is an RNA motif that catalyzes reversible cleavage and ligation reactions at a specific site within an RNA molecule. (Forster et al., Cell, 1987, 49 (2): 211-220). Hammerhead sequence can act by forming a conserved three-dimensional tertiary structure and self-cleaves via a rolling circle replication mechanism. In some embodiments, the HH ribozyme includes the sequence of, for example, CTGATGAGTCCGTGAGGACGAAACGAGTAAGCTCGTC (SEQ ID NO: 2).

A “hepatitis delta virus ribozyme” or “HDV ribozyme”, as used herein, is a non-coding RNA found in the hepatitis delta virus (Ferré-D'Amaré et al., 1998, Nature, 395, pp. 567-574). HDV ribozyme catalyzes cleavage of the phosphodiester bond between the substrate nucleotide or oligonucleotide and the 5-hydroxyl of the ribozyme. HDV ribozyme processes the RNA transcripts to unit lengths in a self-cleavage reaction, which is thought to propagate by a double rolling circle mechanism (Macnaughton et al., Journal of Virology. 76(8): 3920-3927). An HDV ribozyme can contains the sequence of, for example, GGCCGGCATGGTCCCAGCCTCCTCGCTGGCGCCGGCTGGGCAACATGCTTCGGCATG GCGAATGGGAC (SEQ ID NO: 3).

A “polyA tail” or “polyA”, as used herein, is a long chain of adenine nucleotides that is added to a messenger RNA (mRNA) molecule during RNA processing to increase the stability of the molecule. Immediately after a gene in a eukaryotic cell is transcribed, the new RNA molecule undergoes several modifications known as RNA processing. These modifications alter both ends of the primary RNA transcript to produce a mature mRNA molecule. The processing of the 3′ end adds a polyA tail to the RNA molecule. First, the 3′ end of the transcript is cleaved to free a 3′ hydroxyl. Then an enzyme called polyA polymerase adds a chain of adenine nucleotides to the RNA. This process, called polyadenylation, adds a polyA tail that is between 100 and 250 residues long. The polyA tail makes the RNA molecule more stable and prevents its degradation. Additionally, the polyA tail allows the mature messenger RNA molecule to be exported from the nucleus and translated into a protein by ribosomes in the cytoplasm. In specific embodiments, the polyA sequence can be a series of 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 300, or more A nucleotides. In specific embodiments, the polyA sequence is a poyA50 sequence of 50 consecutive A nucleotides. In one specific embodiment, the polyA sequence is atttAAAAAAAAAAAAAAAAAAAAAAAAAAAAAAAAAAAAAAAAAAAAAAAGGATC C (SEQ ID NO: 4). In another specific embodiment, the polyA sequence is atttAAAAAAAAAAAAAAAAAAAAAAAAAAAAAAAAAAAAAAAAAAAAAAAAGGAT CC (SEQ ID NO:5).

In various embodiments, the polynucleotide further comprises elements including, without limitations, polyA, hammerhead ribozyme sequence, Fn direct repeat, hepatitis delta virus ribozyme sequence, or FnHP, or a combination thereof. The additional elements are either to the 5′ end or the 3′ end of the gRNA.

In various embodiments, the polynucleotide is further flanked by transfer DNA. “transfer DNA” or “tDNA”, as used herein, refers to the transferred DNA of the tumor-inducing (Ti) plasmid of some species of bacteria such as Agrobacterium tumefaciens and Agrobacterium rhizogenes (actually an Ri plasmid). The T-DNA is transferred from bacterium into the host plant's nuclear DNA genome (Gelvin, Annual Review of Genetics, 51 (1): 195-217). The T-DNA is bordered by 25-base-pair repeats on each end. Transfer is initiated at the right border and terminated at the left border and requires the vir genes of the Ti plasmid. In various embodiments, the flanking tDNA facilitates the transfer of the polynucleotide into a cell, for example, from an Agrobacterium into a plant cell.

The “marker gene” described herein is a selectable gene which can introduce a trait or traits for the selection of transformed cells or tissues. For example, a marker gene can be an GFP encoding sequence, or a morphogen which enables the transformed cell to divide faster than non-transformed cells and form a visible structure.

A marker gene can be a gene that confers antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Selectable marker genes include genes encoding antibiotic resistance, such as those encoding neomycin phosphotransferase II (NEO), spectinomycin/streptinomycin resistance (AAD or SpcR), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridochromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Accl-S1, Accl-S2 and Accl-S3. Herbicides can also inhibit photosynthesis, including triazine (psbA and 1s+ genes) or benzonitrile (nitrilase gene). Further, such selectable markers can include positive selection markers such as phosphomannose isomerase (PMI) enzyme.

Selectable marker genes can further include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol; methotrexate; hygromycin; spectinomycin; bromoxynil; glyphosate; and phosphinothricin. Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414), red fluorescent protein (DsRFP, RFP, etc), beta-galactosidase, and the maize genes encoding for anthocyanin production (Ludwig, et al., (1990) Science 247:449), and the like (See Sambrook, et al., Molecular Cloning: A Laboratory Manual, Third Edition, Cold Spring Harbor Press, N.Y., 2001), herein incorporated by reference in their entirety. The above list of selectable marker genes is not meant to be limiting. Any reporter or selectable marker gene are encompassed by the present disclosure.

The marker gene can also be any gene of interest according to one of ordinary skill in the art. For example, the marker gene can be a morphogen, an enhancer of homology-directed repair, or an RNAi derivative.

The polynucleotides of interest can be synthesized for optimal expression in a plant. For example, a polynucleotide of interest can have been modified by codon optimization to enhance expression in plants by using codons that are more preferred or more often used in genes natively expressed in plant cells. An insecticidal resistance transgene, an herbicide tolerance transgene, a nitrogen use efficiency transgene, a water use efficiency transgene, a nutritional quality transgene, a DNA binding transgene, a selectable marker transgene/heterologous coding sequence, and/or a nuclease coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. For example, a polynucleotide of interest, e.g., a coding sequence, gene, heterologous coding sequence, or transgene/heterologous coding sequence can be designed to be expressed in plants at a higher level resulting in higher transformation efficiency. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

The expression levels of polynucleotides of interest can be measured by any methods known in the art. For example, polynucleotide expression levels can be measured by quantifying levels of the polynucleotide product, e.g., an RNA or a protein, by, e.g., PCR, real-time PCR, Western blotting, and ELISA. Polynucleotide expression levels can also be assessed by quantifying levels of function of polynucleotide product, for example by quantifying the occurrence of events caused by the polynucleotide product (e.g., morphology and number of regenerated shoots) or by quantifying the levels of product produced by the polynucleotide product, as further disclosed elsewhere in the present disclosure.

A number of promoters may be used in the practice of the disclosure. The promoter may have a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Alternatively, promoters for use in the methods of the present disclosure can be developmentally-regulated promoters. Such promoters may show a peak in expression at a particular developmental stage. Such promoters have been described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Examples of promoters under developmental control include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression constructs comprise a tissue-preferred promoter. A “tissue preferred” promoter is a promoter that initiates transcription mostly, but not necessarily entirely or solely in certain tissues. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

In some embodiments, the expression construct comprises a cell type specific promoter. A “cell type specific” promoter is a promoter that primarily drives expression in certain cell types in one or more organs, for example, embryonic tissue cells. The expression construct can also include cell type preferred promoters. A “cell type preferred” promoter is a promoter that primarily drives expression mostly, but not necessarily entirely or solely in certain cell types in one or more organs, for example, embryonic cells, mesophyll cells, and bundle sheath cells. Such cell-preferred promoters have been described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

Alternatively, promoters for use in the methods of the present disclosure can be promoters that are induced following the application of a particular biotic and/or abiotic stress. Such promoters have been described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

Additionally or alternatively, the promoter can be a spatio-temporal promoter. A “spatio-temporal promoter” as used herein refers to a promoter that is capable of initiating transcription of an operably linked polynucleotide of interest in a spatially, temporally, and/or spatio-temporally specific manner, e.g., in a tissue-specific, an axis-specific, a phase (e.g., developmental phase)-specific, a stage-specific, a timeframe-specific, and/or a timing-specific matter. “Spatio-temporal” transcription initiation as used herein refers to initiation of transcription of an operably linked polynucleotide of interest by a promoter in a spatially, temporally, and/or spatio-temporally specific manner, e.g., in a tissue-specific, an axis-specific, a phase (e.g., developmental phase)-specific, a stage-specific, a timeframe-specific, and/or a timing-specific matter. In some aspects, a spatio-temporal promoter becomes inactive (i.e., does not initiate transcription of an operably linked polynucleotide of interest) in a spatial, temporal, and/or spatio-temporal manner, e.g., outside the desired or designated tissue, axis, phase, stage, timeframe, or timing. The spatio-temporal promoters may produce improved effect on regeneration, development, growth, and/or physiology of plants or plant parts compared to constitutive promoters in expressing certain polynucleotides of interest, particularly when their unregulated or prolonged expression has negative consequences on plant regeneration, development, growth, and/or physiology.

In some aspects, a spatio-temporal promoter can turn itself on and/or off, i.e., initiate transcription in a spatial, temporal, and/or spatio-temporal manner (in a specific tissue, axis, phase, stage, timeframe, and/or timing) without exogenous regulation, and/or becomes inactive (i.e., does not initiate transcription) in a spatial, temporal, and/or spatio-temporal manner (outside a specific tissue, axis, phase, stage, timeframe, and/or timing) without exogenous regulation. Self-regulatory aspects of spatio-temporal promoters of the present disclosure, compared to inducible promoters, can help reduce the skilled labor needed to exogenously regulate activity of the polynucleotides of interest.

The spatio-temporal promoter of the present disclosure can contain the nucleic acid sequence for soybean XCP (e.g., Glyma.04G014800) promoter, soybean DUF1118 (e.g., Glyma.04G161600) promoter, soybean T5AH (e.g., Glyma.18G052400) promoter, pea XCP (e.g., Psat4g084640, Psat5g008960) promoter, medicago XCP (e.g., Medtr3g116080) promoter, pea DUF1118 (e.g., Psat5g207080) promoter, medicago DUF1118 (e.g., Medtr3g026020) promoter, pea T5AH (e.g., Psat5g148400) promoter, medicago T5AH (e.g., Medtr3g467130, Medtr3g467140) promoter, tomato XCP-LIKE (e.g., Solyc12g094700) promoter, Arachis hypogaea XCP-1 (e.g., arahy.Tifrunner.gnm1.ann1.8AM4UR) promoter, Arachis hypogaea XCP-2 (e.g., arahy.Tifrunner.gnm1.ann1.Q7CDUE) promoter, Cicer arietinum XCP-1 (e.g., Ca_04803) promoter, Cicer arietinum XCP-2 (e.g., Ca_17491) promoter, Lupinus albus XCP-1 (e.g., Lalb_Chr23g0265531) promoter, Lotus japonicus XCP-1 (e.g., Lj1g0003774) promoter, Phaseolus acutifolius XCP-1 (e.g., Phacu.CVR.009G145500) promoter, Phaseolus acutifolius XCP-2 (e.g., Phacu.CVR.009G145300) promoter, Phaseolus lunatus XCP-1 (e.g., P109G0000016600.v1) promoter, Phaseolus vulgaris XCP-1 (e.g., Phvu1.009G008200) promoter, Phaseolus vulgaris XCP-2 (e.g., Phvu1.009G008100) promoter, Trifolium pratense XCP-1 (e.g., Tp57577_TGAC_v2_gene38208) promoter, Trifolium pratense XCP-2 (e.g., Tp57577_TGAC_v2_gene15758) promoter, Vigna unguiculata XCP-1 (e.g., Vigun09g263200) promoter, Vigna unguiculata XCP-2 (e.g., Vigun09g263100) promoter, and/or fragments, variants, and combinations thereof.

In some embodiments, the spatio-temporal promoter molecules further comprise a 5′UTR sequence, a 5′UTR intron sequence, an exon sequence from a coding region, and/or an intron sequence from a coding region of the sequence in the plant genome.

In some embodiments, the first promoter operably linked to the marker gene and gRNA coding sequence and the second promoter operably linked to the nucleic acid sequence encoding the nuclease are the same. In other embodiments, the first promoter and the second promoter are different types of promoters. In some embodiments, the first and the second promoter are arranged end-to-end, meaning the two promoters are arranged in opposite directions. In some embodiments, the first promoter is AtUBI11p or AtUBI10p. In some embodiments, the second promoter is AtUBI10p.

### IV. Method of Transforming Plant Cells

In some aspects, the instant disclosure provides a method for transforming a plant cell or a plant tissue. The method includes the steps of: (i) introducing the polynucleotide as described herein into a plant cell or a plant tissue, (ii) culturing the plant cell or the plant tissue, and (iii) selecting for plant cells or plant tissue containing the polynucleotide. In some embodiments, the polynucleotide contains a first promoter operably linked to a marker gene and gRNA coding sequence and a second promoter operably linked to a nucleic acid sequence encoding a nuclease.

The polynucleotide can be introduced into a plant cell or a plant tissue using any standard methods known in the art. For example, the polynucleotide can be introduced into a plant cell, organelle, or plant embryo by a variety of means of transformation, including microinjection (Crossway et al., Biotechniques, 1986, 4:320-334), electroporation (Riggs et al., Proc. Natl. Acad. Sci. USA, 1986, 83:5602-5606), Agrobacterium-mediated transformation (U.S. Pat. Nos. 5,563,055 and 5,981,840), direct gene transfer (Paszkowski et al. (1984) EMBO J. 3:2717-2722), and ballistic particle acceleration [see, for example, U.S. Pat. Nos. 4,945,050; 5,879,918; 5,886,244; and, 5,932,782; Tomes et al., 1995, in Plant Cell, Tissue, and Organ Culture: Fundamental Methods, ed. Gamborg and Phillips (Springer-Verlag, Berlin); McCabe et al., Biotechnology, 1988, 6:923-926); and Lec1 transformation (WO 00/28058). Also see Weissinger et al., Ann. Rev. Genet., 1988, 22:421-477; Sanford et al., Particulate Science and Technology, 1987, 5:27-37 (onion); Christou et al., Plant Physiol., 1988, 87:671-674 (soybean); McCabe et al., Bio/Technology, 1988, 6:923-926 (soybean); Finer and McMullen, In Vitro Cell Dev. Biol., 1991, 27P:175-182 (soybean); Singh et al., Theor. Appl. Genet., 1998, 96:319-324 (soybean); Datta et al., Biotechnology, 1990, 8:736-740 (rice); Klein et al., Proc. Natl. Acad. Sci. USA, 1988, 85:4305-4309 (maize); Klein et al., Biotechnology, 1988, 6:559-563 (maize); U.S. Pat. Nos. 5,240,855; 5,322,783; and, 5,324,646; Klein et al., Plant Physiol., 1988, 91:440-444 (maize); Fromm et al., Biotechnology, 1990, 8:833-839 (maize); Hooykaas-Van Slogteren et al., 1984, Nature (London) 311:763-764; U.S. Pat. No. 5,736,369 (cereals); Bytebier et al., Proc. Natl. Acad. Sci. USA, 1987, 84:5345-5349 (Liliaceae); De Wet et al., 1985, in The Experimental Manipulation of Ovule Tissues, ed. Chapman et al. (Longman, New York), pp. 197-209 (pollen); Kaeppler et al., Plant Cell Reports, 1990, 9:415-418 and Kaeppler et al., Theor. Appl. Genet., 1992, 84:560-566 (whisker-mediated transformation); D'Halluin et al., Plant Cell, 1992, 4:1495-1505 (electroporation); Li et al., Plant Cell Reports, 1993, 12:250-255 and Christou and Ford, Annals of Botany, 1995, 75:407-413 (rice); Osjoda et al., Nature Biotechnology, 1996, 14:745-750 (maize via Agrobacterium tumefaciens)]; all of which are herein incorporated by reference.

Agrobacterium- and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, viral infection, Agrobacterium and viral mediated (Caulimoriviruses, Geminiviruses, RNA plant viruses), liposome mediated and the like. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

The polynucleotide inserted into a plant cell or a plant tissue may be incorporated into the genome of the cell (e.g., nuclear chromosome, plasmid, plastid chromosome or mitochondrial chromosome), converted into an autonomous replicon, or transiently expressed (e.g., transfected mRNA).

After the polynucleotide is introduced into the plant cell or plant tissue, the plant cell or tissue is cultured in accordance with a proper method known in the field (e.g., see McCormick et al., Plant Cell Reports, 1986, 5:81-84) for a proper duration. For example, the plant cell or tissue is cultured for about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 48 hours, about 3 days, about 4 days, about 5 days, about 6 days, or about 7 days. One of ordinary skill in the art can determine the proper duration for culture based on the specific conditions.

Plant cells or plant tissue containing the polynucleotide are selected based on the characteristics provided by the marker gene. For example, in embodiments wherein the marker gene is an antibiotics resistance gene as described herein, the plant cells or plant tissue containing the transformed polynucleotide are selected based on the survival at the presence of an antibiotic corresponding to the antibiotic resistance gene. In some other embodiments, the marker gene is a herbicide resistance gene. Accordingly, the plant cells or plant tissue containing the polynucleotide are selected based on the survival at the presence of corresponding herbicide. In yet some other embodiments, the marker gene can be a green fluorescence protein, a morphogen, an enhancer of HDR, or an RNAi derivative.

A transformed plant cell, or plant tissue may be identified and isolated by selecting or screening the engineered plant material for traits encoded by the marker genes present on the transformed polynucleotide. For instance, selection can be performed by growing the transformed plant cell or plant tissue on media containing an inhibitory amount of the antibiotic or herbicide to which the transforming gene construct confers resistance. Further, transformed plant cell and plant tissue can also be identified by screening for the activities of any visible marker genes (e.g., the 3-glucuronidase, luciferase, or green fluorescent protein genes) that may be present on the recombinant nucleic acid constructs. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay is a method of detecting and quantifying the presence of a DNA sequence. Briefly, a FRET oligonucleotide probe is designed with one oligo within the transgene/heterologous coding sequence and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

A method of detecting and quantifying the presence of a DNA sequence by detecting an amplification reaction can be used. Briefly, the genomic DNA sample comprising the integrated gene expression cassette polynucleotide is screened using a polymerase chain reaction (PCR) based assay. The assay can utilize a PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction.

The confirmation methods include a long read NGS (Next-Generation Sequencing), which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The confirmation methods also include is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The confirmation methods also include a short read technology that uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

A NGS approach can use single DNA molecules for the sequence reactions, e.g., by producing up to 800 million short reads that result in 21 gigabases per run. These reactions are completed using fluorescent dye-labelled virtual terminator nucleotides that is described as a “sequencing by synthesis” approach. A NGS approach can also use a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

In another embodiment, the detection can be completed using blotting assays, including Western blots, Northern blots, and Southern blots. Such blotting assays are commonly used techniques in biological research for the identification and quantification of biological samples. These assays include first separating the sample components in gels by electrophoresis, followed by transfer of the electrophoretically separated components from the gels to transfer membranes that are made of materials such as nitrocellulose, polyvinylidene fluoride (PVDF), or Nylon. Analytes can also be directly spotted on these supports or directed to specific regions on the supports by applying vacuum, capillary action, or pressure, without prior separation. The transfer membranes are then commonly subjected to a post-transfer treatment to enhance the ability of the analytes to be distinguished from each other and detected, either visually or by automated readers.

In a further embodiment the detection can be completed using an ELISA assay, which uses a solid-phase enzyme immunoassay to detect the presence of a substance, usually an antigen, in a liquid sample or wet sample. Antigens from the sample are attached to a surface of a plate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate.

In some embodiments, the gRNA and the marker gene express concurrently. In some embodiments, the gRNA, the marker gene and the nuclease express concurrently. As a result, the fraction of cells with at least one altered target site following introduction of the polynucleotide is increased compared with plant cells following introduction of a control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

“A control polynucleotide”, as used herein, refers to a polynucleotide which does not contain a gRNA that is transcriptionally fused to a marker gene. For example, a control polynucleotide does not contain a single transcription unit (STU) which includes a gRNA and a marker gene. In a specific embodiment, a polynucleotide contains a gRNA operably linked to a first promoter and a marker gene operably linked to a second promoter.

In some embodiments, the plant cell or plant tissue is from an early plant developmental stage. Accordingly, the transformed cell may develop into a homogenously edited plant.

In various embodiments, the plant cell or plant tissue is selected, without limitations, from a group consisting of corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

### V. Method of Altering a Target Site in the Genome of a Plant Cell

In one aspect, the instant disclosure provides a method for altering (e.g., introducing a mutation into) a target site in the genome of a plant cell. The method includes the steps of: (i) introducing a polynucleotide into the plant cell, (ii) culturing the plant cell, and (iii) selecting for plant cells having the polynucleotide. The polynucleotide is any polynucleotide as described herein. In some embodiments, the polynucleotide contains a single gRNA transcriptionally fused to a marker gene along with a gene encoding a nuclease. In other embodiments, the polynucleotide contains more than one gRNA targeting different target sites.

“Altering” a target site, as used herein, refers to introducing at least one mutation including substitution, insertion or deletion, or introducing a single-stranded cut to the genome of a cell, i.e., a plant cell. In some embodiments, the genomic alteration is a point mutation. In other embodiments, the genomic alteration is an insertion of 1 bp, 2 bp, 3 bp, 4 bp, 5 bp, 6 bp, 7 bp, 8 bp, 9 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 160 bp, about 170 bp, about 180 bp, about 190 bp, or about 200 bp. In other embodiments, the genomic alteration is a deletion of 1 bp, 2 bp, 3 bp, 4 bp, 5 bp, 6 bp, 7 bp, 8 bp, 9 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 160 bp, about 170 bp, about 180 bp, about 190 bp, or about 200 bp. In yet some other embodiments, the nuclease has nickase activity, and accordingly the genomic alteration is a single-stranded cleavage. In some embodiments, the polynucleotide contains only a single gRNA, accordingly, only one target site is altered. In other embodiments, the polynucleotide contains more than one gRNA targeting different target sites, accordingly, more than one target sites are altered.

The polynucleotide can be introduced into a plant cell or a plant tissue using any standard technologies known in the art. One of ordinary skill in the art would understand introducing the polynucleotide into a plant cell or a plant tissue means inserting the polynucleotide into the plant cell or cells of the plant tissue, via process such as transfection, transformation or transduction. In some embodiments, the process of introducing the polynucleotide into a plant cell or a plant tissue may involve microorganisms, for example, Agrobacterium. The polynucleotide inserted into a plant cell or a plant tissue may be incorporated into the genome of the cell (e.g., nuclear chromosome, plasmid, plastid chromosome or mitochondrial chromosome), converted into an autonomous replicon, or transiently expressed (e.g., transfected mRNA).

After the polynucleotide is introduced into the plant cell or plant tissue, the plant cell or tissue is cultured in accordance with a proper method known in the field (e.g., see McCormick et al., Plant Cell Reports, 1986, 5:81-84). In some embodiments, the transformed plant cell is cultured for at least about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 48 hours, about 3 days, about 4 days, about 5 days, about 6 days, or about 7 days. One of ordinary skill in the art can determine the sufficient duration for culture based on specific conditions.

Plant cells or plant tissue containing the polynucleotide are selected based on the characteristics provided by the marker gene. For example, the marker gene may be an antibiotic resistance gene as described herein. Accordingly, the plant cells or plant tissue containing the polynucleotide are selected based on the survival at the presence of an antibiotic corresponding to the antibiotic resistance gene. In some other embodiments, the marker gene is a herbicide resistance gene. Accordingly, the plant cells or plant tissue containing the polynucleotide are selected based on the survival at the presence of corresponding herbicide. In yet some other embodiments, the marker gene can be a green fluorescence protein, a morphogen, an enhancer of HDR, or an RNAi derivative.

The nuclease can be any nuclease as described herein. In some embodiments, the nuclease can create a single-stranded cleavage or a double-stranded cleavage. The nuclease of the polynucleotide can be any nuclease described herein. In some embodiments, the cleavage could be a single-strand cleavage or a double-strand cleavage.

A nuclease can be a nickase, an endonuclease, a meganuclease, or a nuclease fusion. For example, a Cas12a (Cpf1) endonuclease coupled with a guide RNA (guide RNA) designed against the genomic sequence of interest can be used (i.e., a CRISPR-Cas12a system). Alternatively, a Cas9 endonuclease coupled with a guide RNA designed against the genomic sequence of interest (a CRISPR-Cas9 system), or a Cms1 endonuclease coupled with a guide RNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al., Nat Rev Microbiol, 2020, 18:67-83)) with their corresponding guide RNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of a polynucleotide, thereby modulating the function of the polynucleotide (Piatek et al., Plant Biotechnol, 2015, J13:578-589). In some embodiments, the nuclease encoded by the coding sequence of the DNA construct is a CRISPR-associated Cas endonuclease. In specific embodiments, the CRISPR nuclease is a Cas12a nuclease, herein used interchangeably with a Cpf1 nuclease. In a specific embodiment, the Cas12a nuclease is a McCpf1 nuclease, e.g., a Mc.2Cpf1 2C-NLS nuclease. In some embodiments, the nuclease is further operably linked to one or more nuclear localization sequences (NLSs) and/or one or more epitope tags. In certain embodiments, the nuclease lacks cleavage activity and is referred to as nuclease dead. In various embodiments, the nuclease has nickase activity. One of ordinary skill in the art can choose any nuclease according the gRNA in order to maximize the editing efficiency. In some preferred embodiments, the nuclease is Cpf1 nuclease.

In certain embodiments, the nuclease lacks cleavage activity and is referred to as nuclease dead. In various embodiments, the nuclease has nickase activity. One of ordinary skill in the art can choose any nuclease according the gRNA in order to maximize the editing efficiency. In some preferred embodiments, the nuclease is Cpf1 nuclease. In some embodiments, the nuclease alters at least one target site of the plant cell. After the polynucleotide is introduced into the plant cell or plant tissue, the nuclease can be expressed and subsequently cleave at least one target site in the genome of the plant cell or the plant tissue. Following cleavage of at least one strand of the target site, at least one mutation can be introduced into the at least one cleaved target site.

In some embodiments, the gRNA and the marker gene express concurrently. In some embodiments, the gRNA, the marker gene, and the nuclease express concurrently. As a result, the fraction of cells with at least one altered target site following introduction of the polynucleotide is increased compared with plant cells following introduction of a control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

As a plant cell can have nuclear, plastid, and mitochondrion genome, the alteration of a target site may happen on nuclear, plastid, and/or mitochondrion genome. Accordingly, in some embodiments, the polynucleotide disclosed herein can contain a nucleic acid sequence encoding a plastid transit peptide or a mitochondrial transit peptide. In some embodiments, the genomic alteration occurs on one allele. In other embodiments, the genomic alteration occurs on both alleles. In one specific embodiment, the genomic alteration occurs at BAS1-6 gene. In another specific embodiment, the genomic alteration occurs PDS1-5 gene. In yet another specific embodiment, the genomic alteration occurs at BS1-1 gene.

In various embodiments, the plant cell or plant tissue is selected, without limitations, from a group consisting of corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

### VI. Method of Increasing the Fraction of Genetically Edited Cells on a Plant Embryo Following Transformation

The instant disclosure further provides a method of increasing the fraction of genetically edited cells in a plant embryo following transformation. The method involves introducing into embryonic plant cells the polynucleotide as described herein, such that the fraction of genetically edited cells following introduction of the polynucleotide is greater than the fraction of genetically edited cells following introduction of a proper control polynucleotide. As used herein, embryonic plant cells or a plant embryo refers to a multicellular structure of undifferentiated plant cells. In specific embodiments, the polynucleotide contains (a) a gRNA transcriptionally fused to a marker gene, and (b) a gene encoding a nuclease, the gRNA and the marker gene are operably linked to a first promoter, and the gene encoding the nuclease is operably linked to a second promoter. As described above, the polynucleotide can be introduced into a plant cell or a plant tissue using any standard technologies known in the art. In various embodiments, the polynucleotide is heterologous to the plant cells.

After the polynucleotide is introduced into the plant cell or plant tissue, the plant cell or tissue is cultured in accordance with a proper method known in the field (e.g., see McCormick et al., Plant Cell Reports, 1986, 5:81-84). The plant cell or plant tissue comprising the polynucleotide disclosed herein can be cultured for a sufficient duration to allow the plant cell or plant tissue to divide and increase in number. In some embodiments, a sufficient duration is about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 48 hours, about 3 days, about 4 days, about 5 days, about 6 days, or about 7 days. One of ordinary skill in the art can determine the sufficient duration for culture based on specific conditions.

In some embodiments, the gRNA and the marker gene express concurrently. In some embodiments, the gRNA, the marker gene and the nuclease express concurrently. As a result, the fraction of genetically edited cells following introduction of the polynucleotide is greater than the fraction of genetically edited cells following introduction of a proper control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%. The increase of the fraction of cells with at least one altered target site can be measured at various time points after the polynucleotide is introduced. In some embodiments, the fraction of cells with at least one altered target site can be measured at about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 48 hours, about 3 days, about 4 days, about 5 days, about 6 days, or about 7 days after the polynucleotide is introduced into the cells.

“Genetically edited”, as used herein, refers to introducing at least one mutation including substitution, insertion or deletion, or introducing a single-stranded cut to the genome of a cell, i.e., a plant cell. In some embodiments, the genetic editing is a point mutation. In other embodiments, the genetic editing is an insertion of 1 bp, 2 bp, 3 bp, 4 bp, 5 bp, 6 bp, 7 bp, 8 bp, 9 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 160 bp, about 170 bp, about 180 bp, about 190 bp, or about 200 bp. In other embodiments, the genetic editing is a deletion of 1 bp, 2 bp, 3 bp, 4 bp, 5 bp, 6 bp, 7 bp, 8 bp, 9 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 160 bp, about 170 bp, about 180 bp, about 190 bp, or about 200 bp. In yet some other embodiments, the nuclease has nickase activity, and accordingly the genetic editing is a single-stranded cleavage.

### VII. A Plant Having the Polynucleotide

The instant disclosure further provides a plant having the polynucleotide, which contains: (a) a gRNA transcriptionally fused to a marker gene, and (b) a gene encoding a nuclease; the gRNA and the marker gene are operably linked to a first promoter, and the gene encoding the nuclease is operably linked to a second promoter. An exemplary polynucleotide is, for example, any one of the constructs in Table 3. In some embodiments, the polynucleotide contains a single gRNA. In other embodiments, the polynucleotide contains more than one gRNAs targeting different target sites.

The polynucleotide can be introduced into the plant cell or a plant tissue using any standard technologies known in the art. One of ordinary skill in the art would understand introducing the polynucleotide into a plant cell or a plant tissue means inserting the polynucleotide into the plant cell or cells of the plant tissue, via process such as transfection, transformation or transduction. In some embodiments, the process of introducing the polynucleotide into a plant cell or a plant tissue may involve microorganisms, for example, Agrobacterium. The polynucleotide inserted into a plant cell or a plant tissue may be incorporated into the genome of the cell (e.g., nuclear chromosome, plasmid, plastid chromosome or mitochondrial chromosome), converted into an autonomous replicon, or transiently expressed (e.g., transfected mRNA).

In some embodiments, all genetically altered cells in the plant are propagated from a first edited cell. In some embodiments, the plant is not a mosaic. In some embodiments, the plant is less mosaic than a control plant having a proper control polynucleotide as described herein.

In some embodiments, the gRNA and the marker gene express concurrently. In some embodiments, the gRNA, the marker gene and the nuclease express concurrently. As a result, the fraction of cells with at least one altered target site following introduction of the polynucleotide is increased compared with plant cells following introduction of a control polynucleotide. In some embodiments, the fraction of cells with at least one altered target site is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

The genome of the plant contains at least one alteration at one or more than one target site. In some embodiments, the at least one alteration at one or more than one target site is introduced by the polynucleotide. In some embodiments, the polynucleotide contains a single gRNA, accordingly, alteration occurs at one target site of the plant cell. In some other embodiments, the polynucleotide contains more than one gRNA targeting different target sites, accordingly, alteration occurs at more than one target site.

In various embodiments, the plant cell or plant tissue is selected, without limitations, from a group consisting of corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Hehanthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of dicots, e.g., legumes.

### EXAMPLES

### Example 1

### Constructs/Plasmids

Constructs generated through standard molecular biology techniques or through external vendor provided synthesis services to produce base plasmids. Guide sequences in STU plasmids were cloned via golden gate technology. Constructs were verified via sanger sequencing after introduction into E. coli or Agrobacterium tumefaciens AGL1 competent cells via chemical methods or electroporation

### Protoplast Transformation

Protoplasts from 2-week old pea shoots were prepared using standard published methodologies from the Jen Sheen lab website with minor modifications. Isolated protoplasts were transformed with plasmid DNA using PEG-mediated transformation. 24-48 hours later protoplast were imaged for GFP expression and later sampled for Edit quantification using ddPCR.

### Soybean Transformation and Genotyping

Transformation was carried out using soybean explants. Explants were co-cultured with acetosyringone treated cultures of Agrobacterium tumefaciens harboring individual constructs. Following a process (10 weeks) of selection steps on media containing antibiotics, selected shoots with or without roots were transferred to plugs in soil in the Greenhouse. Following a 2-week delay in plugs, shoots were sampled and genotyped for copy call and editing via ddPCR and NGS. Editing data was generated for single copy gene targets or dual copy gene targets. Transformation frequency data was also collected.

### Example 2

The instant disclosure uses coordinated expression of nuclease, selectable marker, and guide RNA modules to drive the onset of early editing for giving rise to a highly homogeneous clonal population of cells from which plants can be regenerated. The utility of the instant disclosure is that it allows for reduced construct size, highly-coordinated expression of genes, and the ability to target processes like editing and marker-based selection. This system also allows for the replacement of the marker gene with genes important for morphogenesis, Homology-Directed Repair (HDR), base-editing, or any other gene for editing concepts.

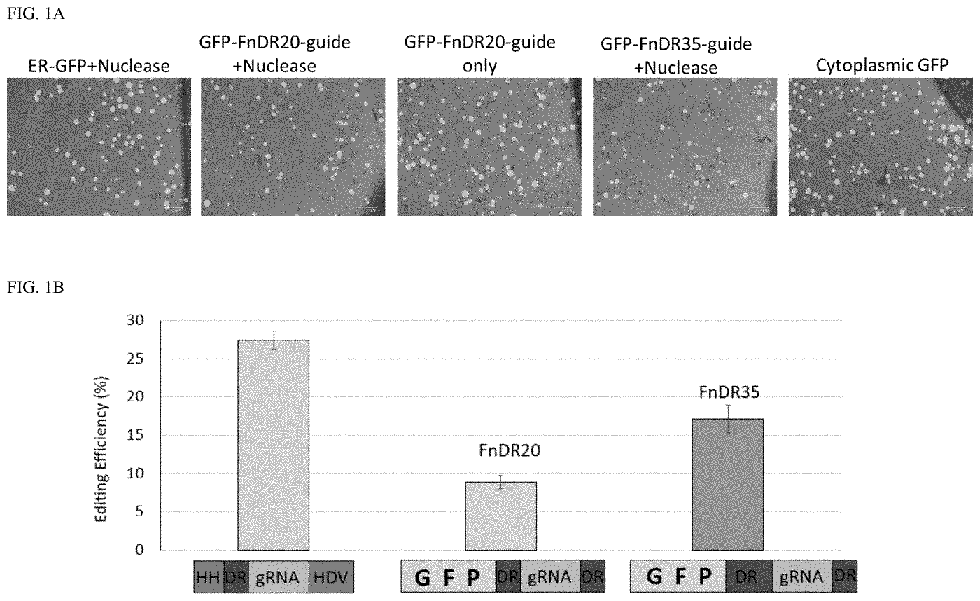

The ability of Cpf1 to process the Direct Repeat (DR)-guide RNA structure has been shown for single and multiple guides (Zetsche et al., Cell, 2015, 163(3): P759-771; Zetsche et al., Nature Biotechnology, 2017, 35, 31-34) allowing it to edit genomic targets at high efficiency. The ability of Cpf1 to process these DR-guide sequences from the context of larger mRNAs was shown later (Zhong et al., Nat Chem Biol., 2017, 13(8): 839-841) in mammalian cells. This concept, shown below, was adapted for use in plant cells at Benson Hill. Constructs where guides expressed as a 3′ extension of an mRNA encoding GFP, when co-delivered with nuclease constructs, lead to both GFP expression (FIG. 1A) and editing in pea protoplasts (FIG. 1B). Guides were expressed with a 5′ Direct Repeat, of either a 20 bp (truncated) or 35 bp (full-length) nature, for evaluating editing efficiency.

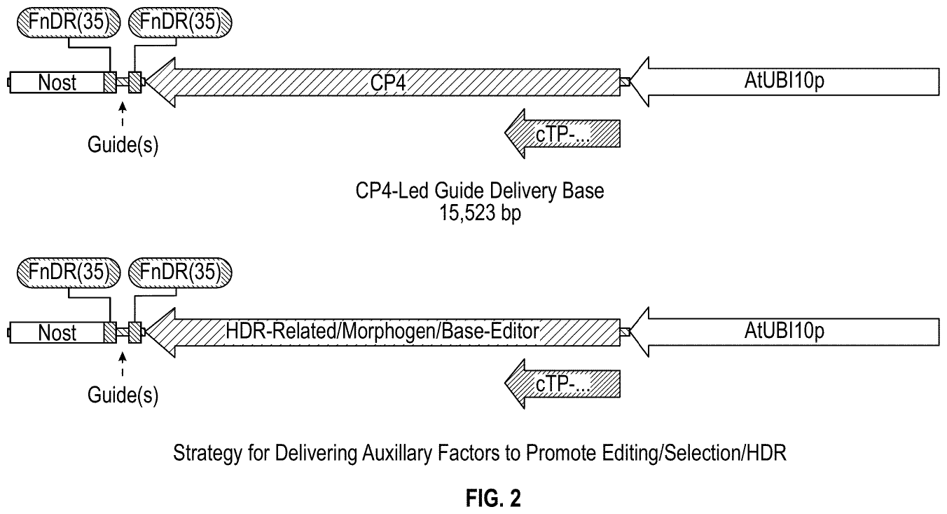

These results indicate that any coding mRNA sequences could be used in lieu of GFP in the guide construct. This premise led to the development of the conceptual idea (FIG. 2) that if guides were expressed as a 3′ extension of a selectable marker gene, the transformed cells that received this construct containing the nuclease and the hybrid selectable marker-guide expressing cassettes will express all three genes-of-interest, the nuclease, the selectable marker, and the guide simultaneously because of the use of constitutive promoters for expressing these elements. Under selection pressure, the first transformed cell will undergo early editing due to a buildup of the editing machinery within this cell because of coordinated expression of the protein and guide RNA elements. This strategy ensures that the first transformed cells in any explant can undergo editing at a high efficiency. Under the selection pressure this cell will then divide and give rise to a clonal population of edited cells, which will ultimately give rise to a transformed & edited shoot. The outcome is a homogenously edited plant, rather than a mosaic comprising unpredictable proportions of edited and unedited cells. Additionally, this strategy ensures that the population of edited plants in relation to transgenic plants is highly-increased.

### Example 3

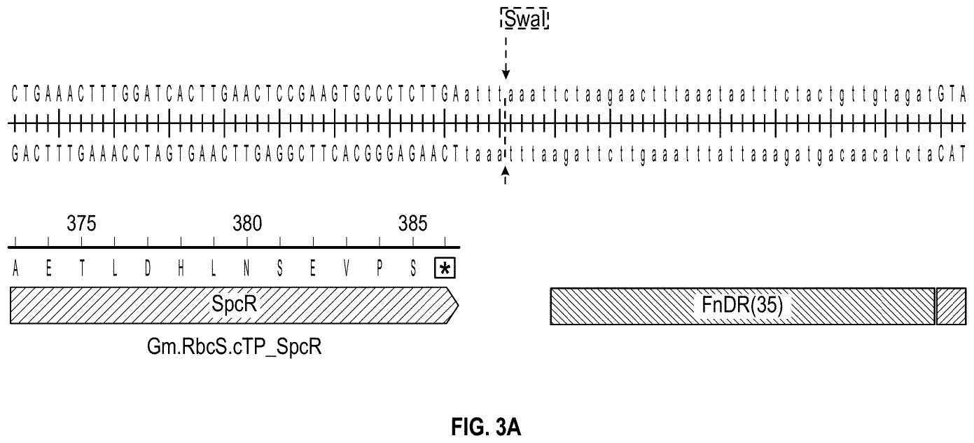

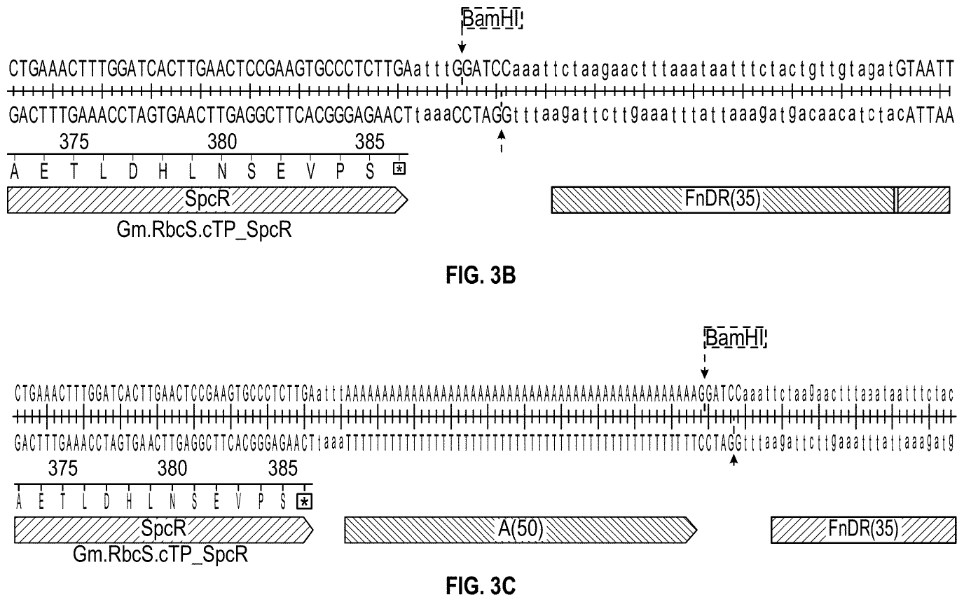

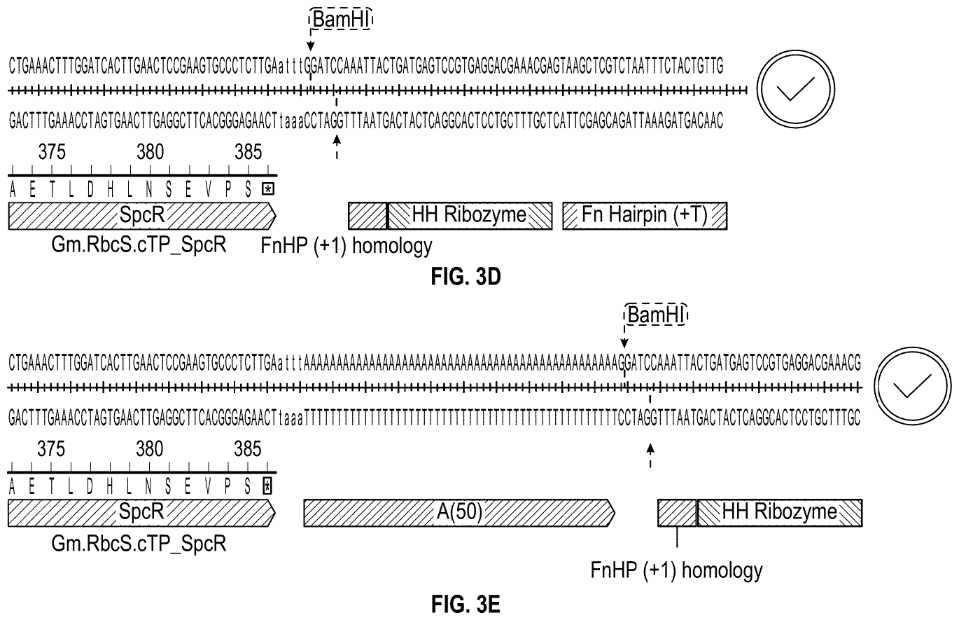

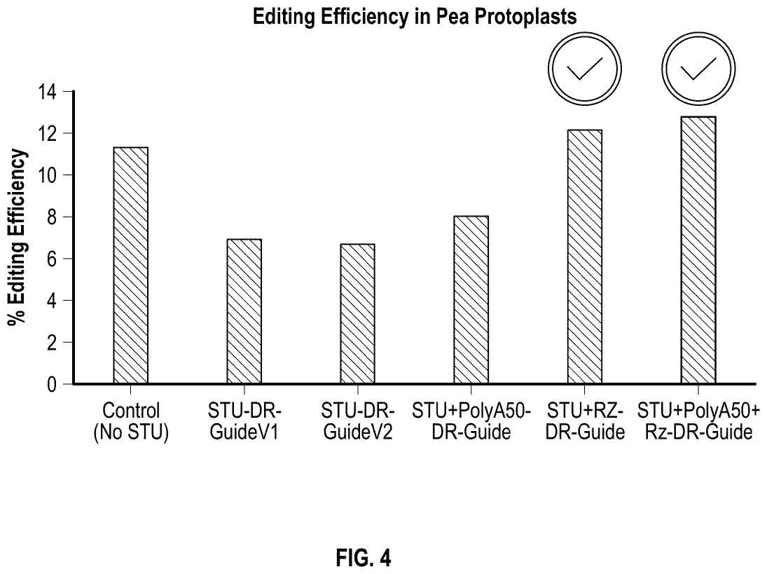

Different versions of the design with or without spacers between the end of the selection marker and the start of the guide (FIGS. 3A-3E) were tested in a pea protoplast system. The plasmids tested included plasmid 134787 (Control (no STU)), plasmid 135856 (STU-DR-GuideV1), plasmid 135885 (STU-DR-GuideV2), plasmid 135886 (STU+PolyA50-DR-Guide), plasmid 135887 (STU+RZ-DR-Guide) and plasmid 135888 (STU+PolyA50+Rz-DR-Guide). (Table 1). It was found that STU designs are equivalent to control/non-STU design for editing in pea protoplasts (Table 1 and FIG. 4)

TABLE 1
Data table for Pea protoplast data.

| | | | | | ddPCR |
|---|---|---|---|---|---|
| | | | | | Editing |
| Plasmid | Note | Descriptor Guide | Plasmid | Descriptor Nuclease | efficiency |
| 134787 | Control | AtUBI11p_FnDR20_Rz_ | 134527 | AtUbi11p_McCpf1_ | 11.39 |
| | (no STU) | PsFAD2B-2 | | Soybean | |
| | | | | 2C-NLS_BsaI | |
| 135856 | STU-DR- | AtUbi10p_Gm.cTP-SpcR-led | 134527 | AtUbi11p_McCpf1_ | 6.96 |
| | GuideV1 | (SwaI)_FnDR(35)_BH257- | | Soybean_2C-NLS_BsaI | |
| | | 2_FnDR(35)_NOSt + | | | |
| | | 2 × 35Sp_GFP-SEKDEL_CaMVt | | | |
| 135885 | STU-DR- | AtUbi10p_Gm.cTP-SpcR-led | 134527 | AtUbi11p_McCpf1_ | 6.73 |
| | GuideV2 | (BamHI)_FnDR(35)_BH257- | | Soybean_2C-NLS_BsaI | |
| | | 2_FnDR(35)_NOSt + | | | |
| | | 2 × 35Sp_GFP-SEKDEL_CaMVt | | | |
| 135886 | STU + Poly | AtUbi10p_Gm.cTP-SpcR- | 134527 | AtUbi11p_McCpf1_ | 8.06 |
| | A50-DR-Guide | led_polyA(48)_FnDR(35)_ | | Soybean_2C-NLS_BsaI | |
| | | BH257-2_FnDR(35)_NOSt + | | | |
| | | 2 × 35Sp_GFP-SEKDEL_CaMVt | | | |
| 135887 | STU + RZ- | AtUbi10p_Gm.cTP-SpcR-led | 134527 | AtUbi11p_McCpf1_ | 12.19 |
| | DR-Guide | (BamHI)_FnDR20_Rz_BH257- | | Soybean_2C-NLS_BsaI | |
| | | 2_NOSt + 2 × 35Sp_GFP- | | | |
| | | SEKDEL_CaMVt | | | |
| 135888 | STU + PolyA50 + | AtUbi10p_Gm.cTP-SpcR- | 134527 | AtUbi11p_McCpf1_ | 12.85 |
| | Rz-DR-Guide | led_polyA(48)_FnDR20_Rz_ | | Soybean_2C-NLS_BsaI | |
| | | BH257-2_NOSt + 2 × 35Sp_ | | | |
| | | GFP-SEKDEL_CaMVt | | | |
| 134787 | Control | AtUBI11p_FnDR20_Rz_ | 134770 | AtUBI11p_MAD7- | 7.39 |
| | (no STU) | PsFAD2B-2 | | 2C-Gm | |
| 135886 | STU + Poly | AtUbi10p_Gm.cTP-SpcR- | 134770 | AtUBI11p_MAD7- | 2.59 |
| | A50-DR-Guide | led_polyA(48)_FnDR(35)_ | | 2C-Gm | |
| | | BH257-2_FnDR(35)_NOSt + | | | |
| | | 2 × 35Sp_GFP-SEKDEL_CaMVt | | | |
| 135888 | STU + Poly A50 + | AtUbi10p_Gm.cTP-SpcR- | 134770 | AtUBI11p_MAD7- | 6.21 |
| | Rz-DR-Guide | led_polyA(48)_FnDR20_Rz_ | | 2C-Gm | |
| | | BH257-2_NOSt + 2 × 35Sp_ | | | |
| | | GFP-SEKDEL_CaMVt | | | |

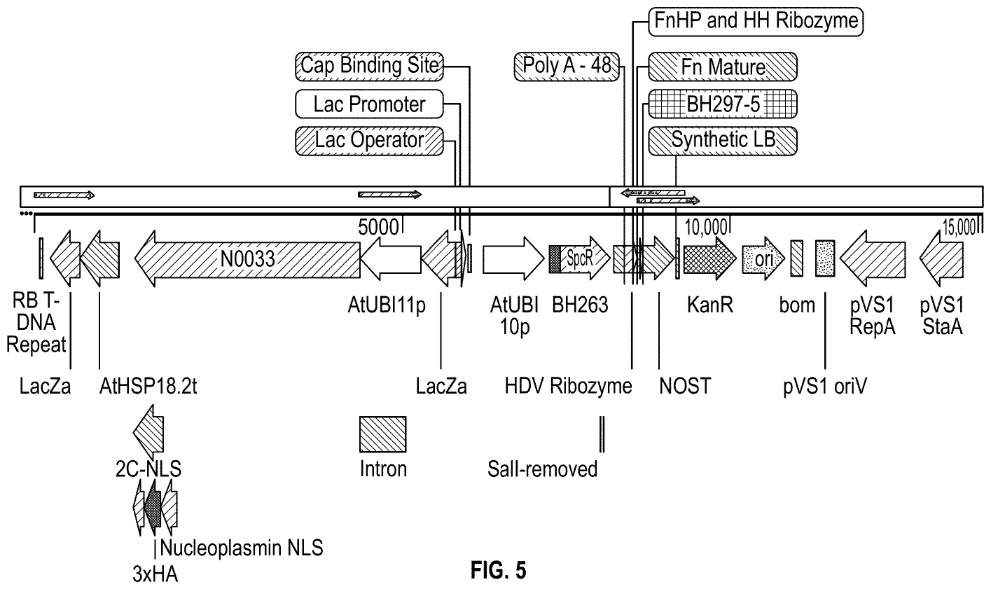

Next, T-DNA vectors were made based on STU+PolyA50+Rz-DR-Guide design to transform soybean, some promoters were changed in this design. An exemplary vector of this design is construct 136008 (FIG. 5), which has a guide RNA targeting PDSg5 as a 3′ extension to the spectinomycin resistance gene. The editing efficiency of construct 136008 in planta was measured (Table 2). Sample numbers were restricted to 44 and the constructs used an older nuclease version to demonstrate the efficiency of the constructs.

The edited region was sequenced. According to Table 2, it was clear that 10/18 events had >25% editing efficiency; 7/18 events had >40% editing efficiency, and 5/18 events had >70% editing efficiency. This high percentage of editing efficiency was significant to enable heritable edits.

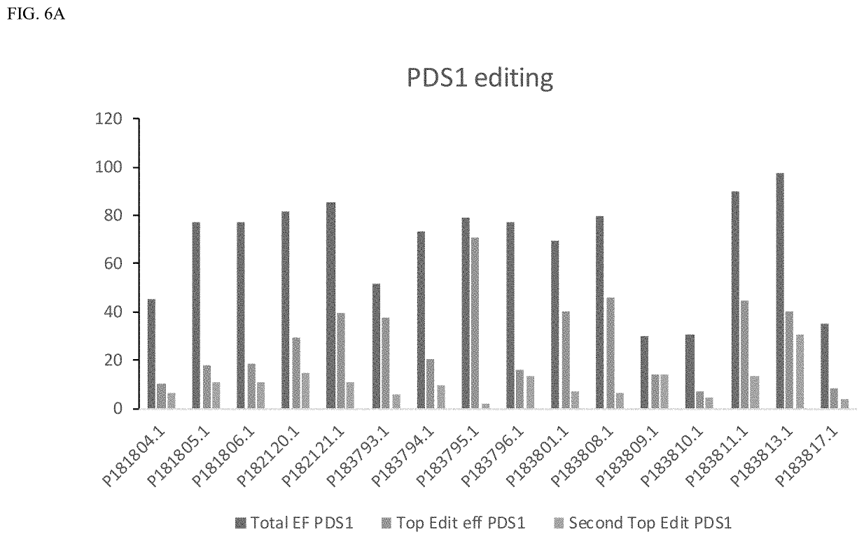

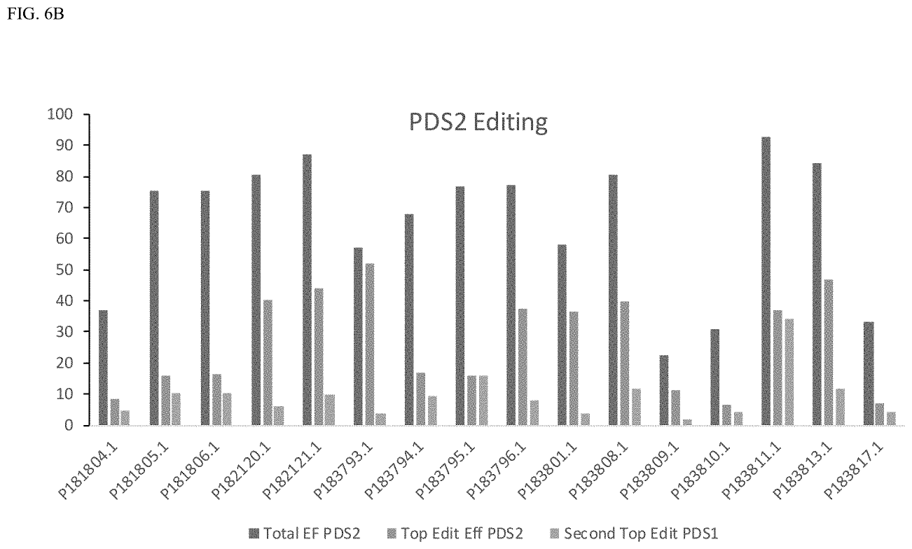

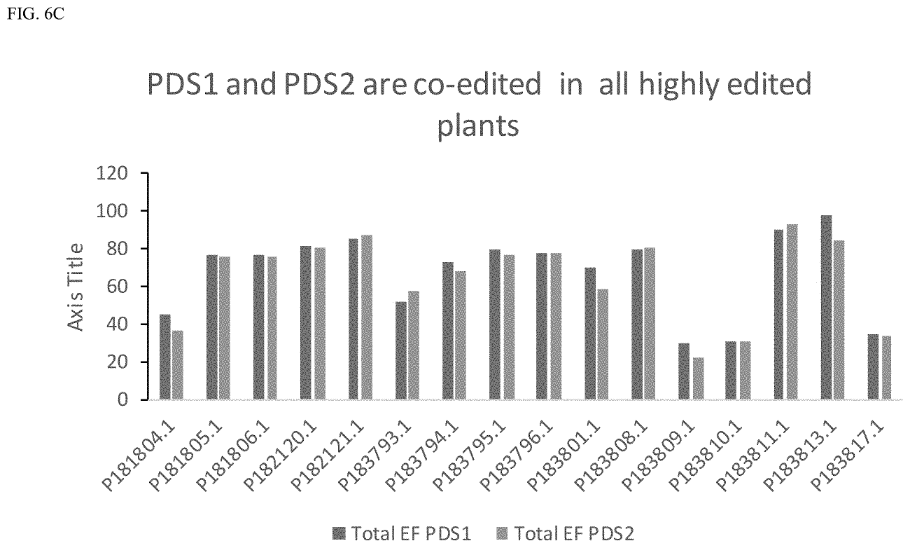

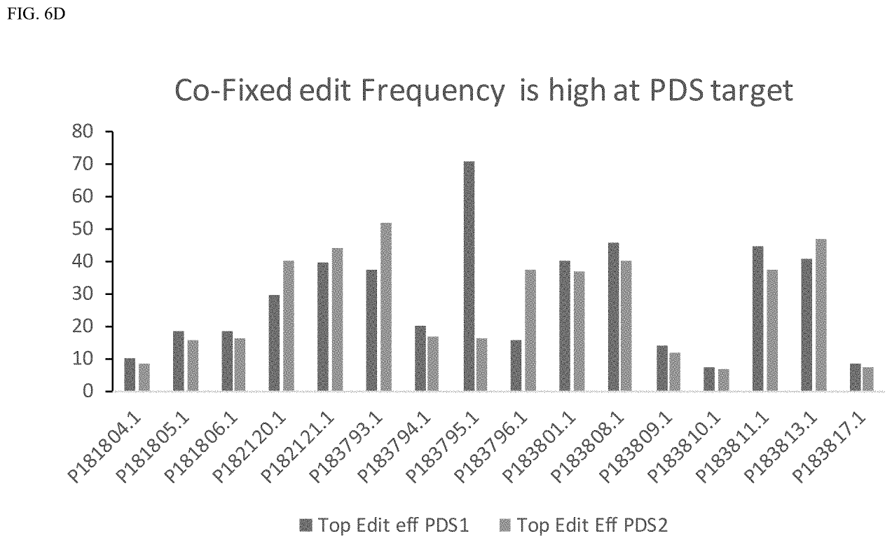

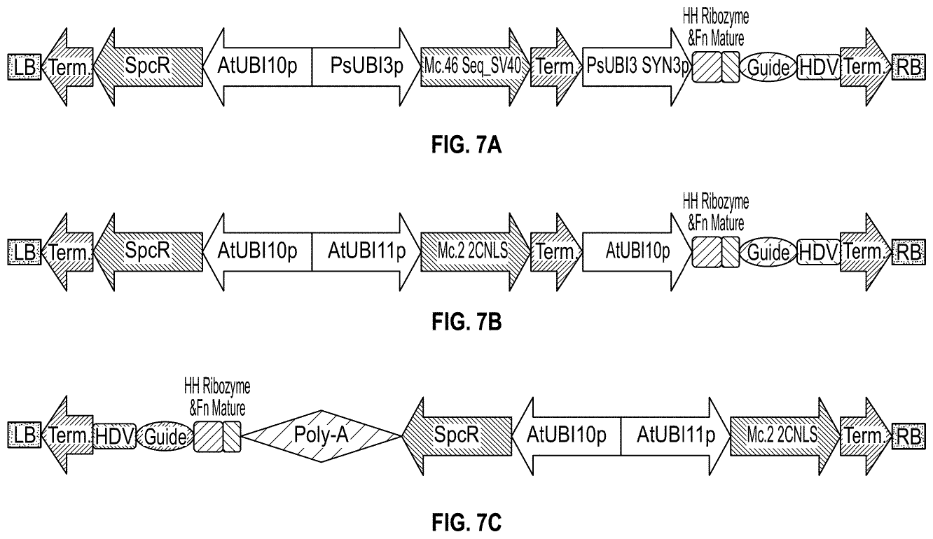

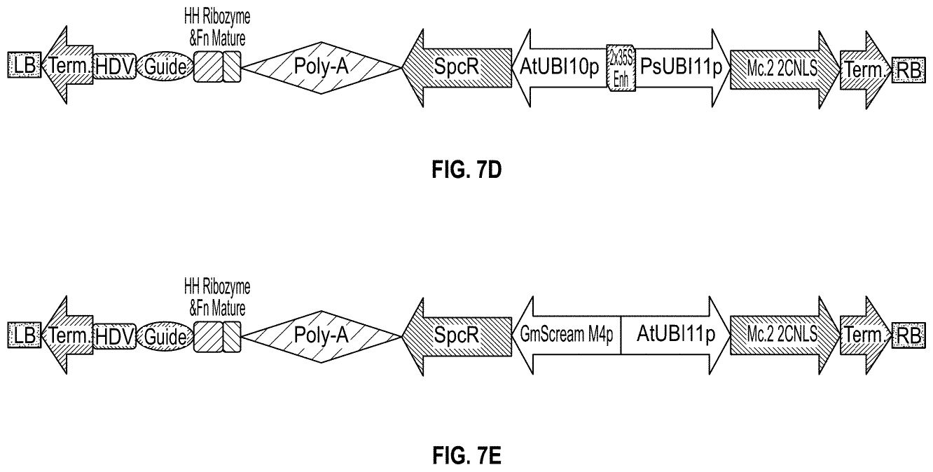

In addition, Next Generation Sequencing (NGS) data indicating the total editing efficiency of the first copy of PDS gene (PDS1), the top edit efficiency of PDS1 and the second top edit efficiency of PDS1 were provided (FIG. 6A). NGS data showing the total editing efficiency of the second copy of PDS gene (PDS2), top edit efficiency of PDS2 and the second top edit efficiency of PDS1 were provided (FIG. 6B). NGS data showed that the PDS1 and PDS2 were co-edited in all highly edited plants (FIG. 6C), and co-fixed edit frequency was high at PDS target (FIG. 6D).

TABLE 2
In planta editing data at PDSg5 with SPCR-led guide construct in T0 events.

| | Plant Pedigree | | | | Inoculation | | | ddPCR |
|---|---|---|---|---|---|---|---|---|
| Name | Display Name | Sel Marker | SPCR | Constructs | Reagents | BH IDs | Targets | EF % |
| P181803.1 | P181803 | SPCR | 1 | 136008 | GE0894 | BH297-5 | PDS1-5 | 72.99 |
| P181804.1 | P181804 | SPCR | >2 | 136008 | GE0894 | BH297-5 | PDS1-5 | 42.96 |
| P181805.1 | P181805 | SPCR | 1 | 136008 | GE0894 | BH297-5 | PDS1-5 | 76.81 |
| P181806.1 | P181806 | SPCR | UNDET | 136008 | GE0894 | BH297-5 | PDS1-5 | 27.61 |
| P182110.1 | P182110 | SPCR | UNDET | 136008 | GE0894 | BH297-5 | PDS1-5 | 1.15 |
| P182111.1 | P182111 | SPCR | UNDET | 136008 | GE0894 | BH297-5 | PDS1-5 | 12.18 |
| P182112.1 | P182112 | SPCR | UNDET | 136008 | GE0894 | BH297-5 | PDS1-5 | 8.44 |
| P182114.1 | P182114 | SPCR | UNDET | 136008 | GE0894 | BH297-5 | PDS1-5 | 0 |
| P182115.1 | P182115 | SPCR | 1 | 136008 | GE0894 | BH297-5 | PDS1-5 | 1.55 |
| P182116.1 | P182116 | SPCR | >2 | 136008 | GE0894 | BH297-5 | PDS1-5 | 1.08 |
| P182117.1 | P182117 | SPCR | UNDET | 136008 | GE0894 | BH297-5 | PDS1-5 | 24.55 |
| P182118.1 | P182118 | SPCR | 1 | 136008 | GE0894 | BH297-5 | PDS1-5 | 0 |
| P182119.1 | P182119 | SPCR | UNDET | 136008 | GE0894 | BH297-5 | PDS1-5 | 22.08 |
| P182120.1 | P182120 | SPCR | 1 | 136008 | GE0894 | BH297-5 | PDS1-5 | 83.69 |
| P182122.1 | P182122 | SPCR | 2 | 136008 | GE0894 | BH297-5 | PDS1-5 | 83.56 |
| P182109.1 | P182109 | SPCR | UNDET | 136008 | GE0894 | BH297-5 | PDS1-5 | 13.39 |
| P182113.1 | P182113 | SPCR | 2 | 136008 | GE0894 | BH297-5 | PDS1-5 | 46.79 |
| P182121.1 | P182121 | SPCR | 1 | 136008 | GE0894 | BH297-5 | PDS1-5 | 88.62 |

### Example 4

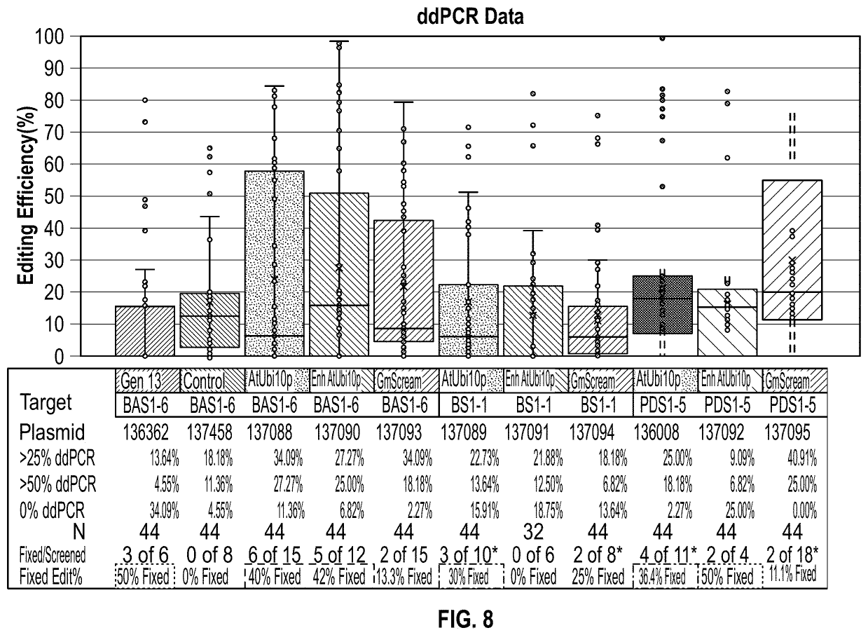

Next, an expanded test of 11 constructs (Table 3) for the editing efficiency at three targets including PDS1-g5, BAS1-g6 and BS1-g1 were performed. The designs of each constructs were detailed at FIGS. 8A-8E. Sample numbers were restricted to 44 and the constructs used an older nuclease version to demonstrate the efficiency of the constructs.

TABLE 3
Construct Summary for Soy Plant Data

| Plasmid | Nuclease | Guide | Note |
|---|---|---|---|
| 136362 | PsUBI3p Mc.46 | PsUBI3 SYN3p FnDR20 Rz | Gen 13 Non- |
| | SEQ-SV40 HSPt | BAS1-6 NOSt | STU Control |
| 137458 | AtUBI11p Mc.2 | AtUBI1Op FnDR20 Rz | Gen 8 non- |
| | 2C-NLS HSPt | BAS1-6 NOSt | STU Control |
| 137088 | AtUBI11p Mc.2 | AtUBI10p SpcR PolyA Rz | STU |
| | 2C-NLS HSPt | BAS1-6 NOSt | |
| 137089 | AtUBI11p Mc.2 | AtUBI10p SpcR Poly A Rz | STU |
| | 2C-NLS HSPt | BS1-1 NOSt | |
| 136008 | AtUBI11p Mc.2 | AtUBI10p SpcR PolyA Rz | STU |
| | 2C-NLS HSPt | PDS1-5 NOSt | |
| 137090 | AtUBI11p Mc.2 | 2 × 35S Enh AtUBI10p | STU |
| | 2C-NLS HSPt | SpcR Poly A Rz BAS1-6 | |
| | | NOSt | |
| 137091 | AtUBI11p Mc.2 | 2 × 35S Enh AtUBI10p | STU |
| | 2C-NLS HSPt | SpcR Poly A Rz BS1-1 | |
| | | NOSt | |
| 137092 | AtUBI11p Mc.2 | 2 × 35S Enh AtUBI10p | STU |
| | 2C-NLS HSPt | SpcR Poly A Rz PDS1-5 | |
| | | NOSt | |
| 137093 | AtUBI11p Mc.2 | GmScream M4p SpcR | STU |
| | 2C-NLS HSPt | Poly A Rz BAS1-6 NOSt | |
| 137094 | AtUBI11p Mc.2 | GmScream M4p SpcR | STU |
| | 2C-NLS HSPt | Poly A Rz BS1-1 NOSt | |
| 137095 | AtUBI11p Mc.2 | GmScream M4p SpcR | STU |
| | 2C-NLS HSPt | Poly A Rz PDS1-5 NOSt | |

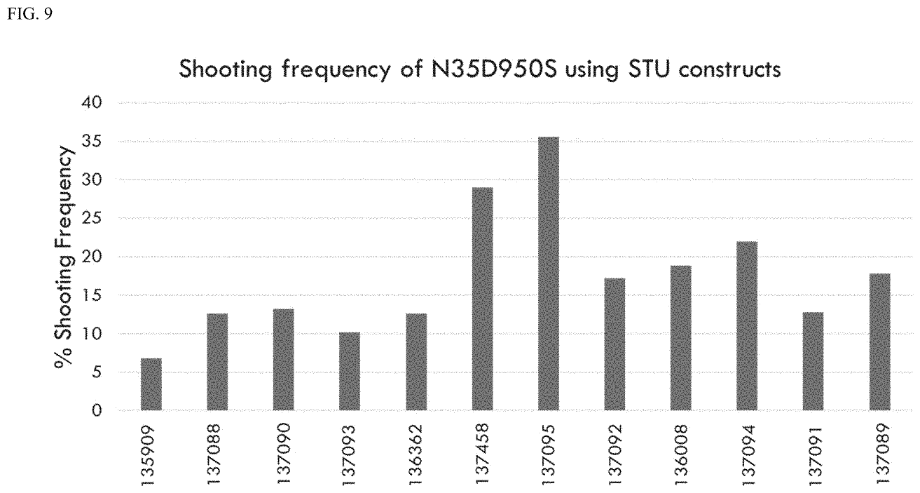

As concluded from FIGS. 9 and 10, and Tables 4-6, there was high frequency of samples showing the highly desirable fraction of edits known as pre-dominant edits which were inheritable edits. In addition, there was high frequency of plants which were edited at two copies of the target gene. Mono and Bi-allelic edits were obtained through this technology at high frequency. Moreover, low sample number and use of a lower gen nuclease showed the efficacy of the technology.

TABLE 4
NGS-based Genotyping data for BAS1g6.

| Construct/ | | | | Pre- | | Mono/Bi- |
|---|---|---|---|---|---|---|
| Target | Construct | Total # of | Samples | dominant | Bi-allelic | Allelic |
| BAS1g6 | Type | samples | for NGS | Edits | edits | frequency |
| 137088 | STU | 44 | 15 | 6 | 1 | 13.6%/2.25% |
| 137090 | STU | 44 | 12 | 5 | 0 | 11.3%/0% |
| 137093 | STU | 44 | 15 | 2 | 0 | 4.5%/ 0% |
| 136362 | Control | 44 | 6 | 3 | 0 | 6.8%/0% |
| 137458 | Control | 44 | 8 | 0 | 0 | 0/0 |

TABLE 5
NGS-based Genotyping data for PDS1g5.

| | | | | Pre-dominant | | Pre-Dominant | | | Dual |
|---|---|---|---|---|---|---|---|---|---|
| Construct/ | | Total | | Edits PDS1 | | Edits PDS2 | | Both | target |
| Target | Construct | # of | Samples | Pre- | | Pre- | | Copy | editing |
| PDS1g5 | Type | samples | for NGS | Dom | BiAllelic | Dom | BiAllelic | Edited | frequency |
| 137095 | STU | 44 | 18 | 4 | 0 | 3 | 1 | 2 | 4.5% |
| 137092 | STU | 44 | 4 | 2 | 0 | 2 | 0 | 1 | 2.25% |
| 136008 | STU | 44 | 11 | 5 | 0 | 2 | 1 | 2 | 4.5% |

TABLE 6
NGS-based Genotyping data for BS1g1.

| | | | | Pre-dominant | | Pre-Dominant | | | Dual |
|---|---|---|---|---|---|---|---|---|---|
| Construct/ | | Total | Sample | Edits BS1 | | Edits BS2 | | Both | target |
| Target | Construct | # of | # to | Pre- | | Pre- | | Copy | editing |
| BS1g1 | Type | samples | NGS | Dom | BiAllelic | Dom | BiAllelic | Edited | frequency |
| 137094 | STU | 44 | 8 | 3 | 1 | 1 | 1 | 1 | 2.25% |
| 137089 | STU | 44 | 10 | 3 | 0 | 2 | 0 | 1 | 2.25% |
| 137091 | STU | 32 | 7 | 0 | 0 | 0 | 0 | 0 | 0 |

1. A polynucleotide comprising:
(a) a gRNA transcriptionally fused to a marker gene, and
(b) a gene encoding a nuclease;
wherein the gRNA and the marker gene are operably linked to a first promoter, and the gene encoding the nuclease is operably linked to a second promoter.

2. The polynucleotide of claim 1, wherein the gRNA is operably linked to the 3′ extension of marker gene.

3. The polynucleotide of claim 1, wherein the polynucleotide further comprises a spacer between the marker gene and the gRNA.

4. The polynucleotide of claim 3, wherein the spacer comprises a polyA, an HH ribozyme, an FnHP, or an Fn direct repeat, or a combination thereof.

5. (canceled)

6. The polynucleotide of claim 1, wherein said marker gene is a spectinomycin resistance gene.

7.-8. (canceled)

9. The polynucleotide of claim 1, wherein the nuclease is a Cpf1 nuclease.

10. (canceled)

11. The polynucleotide of claim 1, wherein the polynucleotide comprises any one of the constructs of Table 3.

12. A method of transforming a plant cell comprising the steps of:
(i) introducing the polynucleotide of claim 1 into a plant cell;
(ii) culturing the plant cell; and
(iii) selecting for plant cells comprising the polynucleotide.

13. The method of claim 12, wherein selecting in step (iii) comprises selecting for plant cells based on marker gene expression.

14. (canceled)

15. The method of claim 12, wherein the plant cell is from an early plant developmental stage.

16.-19. (canceled)

20. A method of altering a target site in the genome of a plant cell, comprising the steps of:
(i) introducing the polynucleotide of claim 1 into the plant cell;
(ii) culturing the plant cell for a sufficient duration;
(iii) selecting for plant cells comprising the polynucleotide;
wherein the target site is altered.

21. (canceled)

22. The method of claim 20, wherein selecting in step (iii) comprises selecting for plant cells based on marker gene expression.

23. (canceled)

24. The method of claim 20, wherein the marker gene, the gRNA and the nuclease express concurrently.

25.-27. (canceled)

28. A method of increasing the fraction of genetically edited cells in a plant embryo following transformation, said method comprising introducing into the plant embryo a polynucleotide comprising:
(a) a gRNA transcriptionally fused to a marker gene, and
(b) a gene encoding a nuclease;
wherein the gRNA and the marker gene are operably linked to a first promoter, and the gene encoding the nuclease is operably linked to a second promoter.
wherein the fraction of genetically edited cells following introduction of said polynucleotide is greater than the fraction of genetically edited cells following introduction of a proper control polynucleotide.

29. (canceled)

30. The method of claim 28, wherein the marker gene, the gRNA, and the nuclease express concurrently.

31. The method of claim 28, wherein the fraction of genetically edited cells following introduction of said polynucleotide is increased by at least 5%, 10%, 20%, 30%, 40%, 50%, 60%, 70%, 80%, 90%, 100%, 200%, 300%, 400%, 500%, 600%, 700%, 800%, 900%, or at least 1000%.

32. (canceled)

33. A plant comprising a polynucleotide comprising:
(a) a gRNA transcriptionally fused to a marker gene, and
(b) a gene encoding a nuclease;
wherein the gRNA and the marker gene are operably linked to a first promoter, and the gene encoding the nuclease is operably linked to a second promoter.

34. The plant of claim 33, wherein the genome of the plant comprises an alteration at a target site.

35.-36. (canceled)

37. The plant of claim 33, wherein the fraction of cells with at least one alteration at a target site is increased compared with a plant comprising a proper control polynucleotide.

38. (canceled)

39. The plant of claim 33, wherein the plant is selected from a group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.
